(19) United States
(12) Patent Application Publication
(10) Pub. No.: US 2009/0286944 A1
(43) Pub. Date: Nov. 19, 2009

(54) SELECT PHENOL-HETEROCYCLE LIGANDS, METAL COMPLEXES FORMED THEREFROM, AND THEIR USES AS CATALYSTS

(75) Inventors: Lily J. Ackerman, San Francisco, CA (US); Margarete K. Leclerc, Mountain View, CA (US); Xining Luo, Sunnyvale, CA (US); James A.W. Shoemaker, Gilgory, CA (US); Pu Sun, San Jose, CA (US); Jessica Zhang, Milpitas, CA (US)

Correspondence Address: DERICK E. ALLEN (28217);ARMSTRONG TEASDALE LLP, ONE METROPOLITAN SQUARE, SUITE 2600, ST. LOUIS, MO, 63102-2740, US

(73) Assignee: SYMYX TECHNOLOGIES, INC., Sunnyvale, CA (US)

(21) Appl. No.: 12/121,391

(22) Filed: May 15, 2008

(51) Int. Cl. C08F 4/44; C07D 271/06; B01J 31/18

(52) U.S. Cl. 526/147; 548/106; 502/167

(57) ABSTRACT

Select phenol-heterocycle ligands, metal-ligand compositions or complexes formed therefrom, and their use as catalysts in polymerization reactions and other transformations are disclosed herein. The catalysts have high performance characteristics, including the ability to catalyze reactions at high temperatures. The catalysts are particularly well-suited for the polymerization of olefins, including the polymerization of styrene to form polystyrene.

### TECHNICAL FIELD

The present invention relates to select phenol-heterocycle ligands, metal-ligand compositions or complexes formed therefrom, and their use as catalysts in polymerization reactions and other transformations.

### BACKGROUND OF THE INVENTION

Ancillary (or spectator) metal-ligand coordination complexes (including organometallic complexes) and compositions are useful as catalysts, additives, stoichiometric reagents, solid-state precursors, therapeutic reagents and drugs. Ancillary metal-ligand coordination complexes of this type can be prepared by combining an ancillary ligand with a suitable metal compound or metal precursor in a suitable solvent at a suitable temperature. The ancillary ligand contains functional groups that bind to the metal center(s), remain associated with the metal center(s), and therefore provide an opportunity to modify the steric, electronic and chemical properties of the active metal center(s) of the complex.

One example of the use of these types of ancillary metal-ligand complexes and compositions is in the field of polymerization catalysis. In connection with single site catalysis, the ancillary ligand typically offers opportunities to modify the electronic and/or steric environment surrounding an active metal center. This allows the ancillary ligand to assist in the creation of possibly different polymers. More recently, interest has expanded into the next generation of non-cyclopentadienyl catalysts for olefin polymerization. (See, e.g., U.S. Pat. No. 5,318,935 and PCT Application No. WO 99/05186. See also, related U.S. patent application Ser. No. 11/305,426, filed Dec. 16, 2005, and published as U.S. 2006-0135713.)

Despite the efforts of many workers in the field, a need remains for commercially suitable catalyst systems for the polymerization of monomers, and in particular for the homopolymerization or copolymerization of vinylidene aromatic monomers, especially styrene or substituted styrenes, for the production of polymers having molecular weights high enough for general commercial use, and/or variable tacticities, at high reaction temperatures. In particular, what is needed is a catalyst or family of catalysts capable of making a range of polymers (e.g., aromatic polymers, such as vinylidene aromatic polymers) with differing degrees of stereoregularity that can be controlled by the appropriate choice of catalyst and conditions. Desirably, such a catalyst would provide: (i) greater polymerization activity, measured for example by the amount of polymer (e.g., mg of polymer) formed per unit time (e.g., minutes) relative to the amount of catalyst (e.g., μmoles of catalyst) used; (ii) a higher molecular weight (Mw or Mn) of the polymer; (iii) a more narrow polydispersity (PDI) of the polymer; (iv) a polymer having a range of stereo-sequence distributions; and/or (v) the ability to conduct the polymerization reaction at a high temperature (e.g., greater than about 120° C.). A range of product opportunities could then exist, including the formation of polymers uniquely suited for preparation via high temperature solution polymerization processes.

### BRIEF SUMMARY OF THE INVENTION

The invention features ligands comprising a phenol ring and a heterocyclic ring (i.e., phenol-heterocycle-based ligands), metal-ligand compositions or complexes formed therefrom, and their use as catalysts in polymerization reactions (e.g., the polymerization of olefins) and other transformations, as well as methods for preparing these ligands and for using the compositions or complexes in catalytic transformations (such as olefin polymerization). In particular, the ligands have a 3-phenol-oxadiazole-based structure (i.e., the ligands comprise a phenol ring and an oxadiazole ring), and more particularly may have a 3-phenol-1,2,4-oxadiazole structure, as will be discussed in more detail below. Catalysts according to the invention can be provided as compositions including a ligand, a metal precursor, and optionally an activator, a combination of activators, or an activator technique. Alternatively, catalysts can be provided by metal-ligand complexes and optionally may additionally include an activator, a combination of activators or an activator technique.

Accordingly, in one aspect, the present invention is directed to a compound that is a metal ligand complex characterized by the general formula:

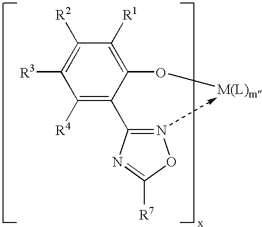

wherein: (a) each of R¹, R², R³ and R⁴ are the same or different and are independently selected from the group consisting of hydrogen, alkyl, substituted alkyl, cycloalkyl, substituted cycloalkyl, heteroalkyl, substituted heteroalkyl, heterocycloalkyl, substituted heterocycloalkyl, aryl, substituted aryl, heteroaryl, substituted heteroaryl, alkoxy, aryloxy, halo, silyl, boryl, phosphino, amino, thioalkyl, thioaryl, nitro, and combinations thereof, with the proviso that (i) at least one of R¹, R², R³ and R⁴ is selected from the group consisting of carbazolyl and substituted carbazolyl; and further (ii) at least one of R¹, R², R³ and R⁴ is selected from the group consisting of alkyl, substituted alkyl, halo, and alkoxy; (b) R⁷ is selected from the group consisting of phenyl, substituted phenyl, and anthracenyl; (c) M is a metal selected from the group consisting of groups 3 through 6 of the periodic table elements and lanthanides; (d) each L is independently selected from the group consisting of hydrogen, halogen, optionally substituted alkyl, heteroalkyl, allyl, diene, alkenyl, heteroalkenyl, alkynyl, heteroalkynyl, aryl, heteroaryl, alkoxy, aryloxy, boryl, silyl, amino, phosphino, ether, thioether, phosphine, amine, carboxylate, alkylthio, arylthio, 1,3-dionate, oxalate, carbonate, nitrate, sulphate, and combinations thereof; (e) x is 1, 2, 3, or 4; and (f) m″ is 0, 1, 2, 3, or 4.

In particular, the present invention is directed to a compound as set forth above, wherein the metal ligand complex is characterized by a formula:

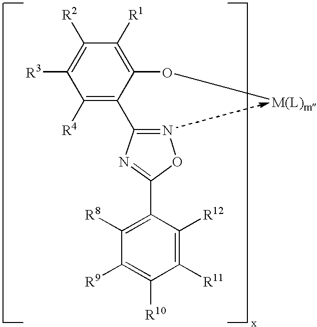

wherein: (a) R¹, R², R³ and R⁴ are as defined and provided for above; (b) R⁸, R⁹, R¹⁰, R¹¹, and R¹² are the same or different and are independently selected from the group consisting of hydrogen, alkyl, substituted alkyl, cycloalkyl, substituted cycloalkyl, heteroalkyl, substituted heteroalkyl, heterocycloalkyl, substituted heterocycloalkyl, aryl, substituted aryl, heteroaryl, substituted heteroaryl, alkoxy, aryloxy, halo, silyl, boryl, phosphino, amino, thioalkyl, thioaryl, nitro, and combinations thereof, and optionally with two or more of R⁸, R⁹, R¹⁰, R¹¹, and R¹² being joined to form a fused ring system having up to 50 atoms, not counting hydrogen atoms.

In another aspect, the present invention is directed to a composition comprising: (a) a compound characterized by the general formula:

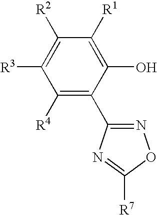

wherein: (a) each of R¹, R², R³ and R⁴ are the same or different and are independently selected from the group consisting of hydrogen, alkyl, substituted alkyl, cycloalkyl, substituted cycloalkyl, heteroalkyl, substituted heteroalkyl, heterocycloalkyl, substituted heterocycloalkyl, aryl, substituted aryl, heteroaryl, substituted heteroaryl, alkoxy, aryloxy, halo, silyl, boryl, phosphino, amino, thioalkyl, thioaryl, nitro, and combinations thereof, with the proviso that (i) at least one of R¹, R², R³ and R⁴ is selected from the group consisting of carbazolyl and substituted carbazolyl; and further (ii) at least one of R¹, R², R³ and R⁴ is selected from the group consisting of alkyl, substituted alkyl, halo, and alkoxy, and R⁷ is selected from the group consisting of phenyl, substituted phenyl, and anthracenyl; and (b) a metal precursor characterized by the general formula M(L)ₙ, where M is a metal selected from groups 3-6 of the Periodic Table of Elements and Lanthanide elements of the Periodic Table of Elements, each L is a moiety that forms a covalent, dative or ionic bond with M, and n is 1, 2, 3, 4, 5, or 6.

In particular, the present invention is directed to a composition as set forth above, wherein the compound in part (a) is characterized by a formula selected from the group consisting of

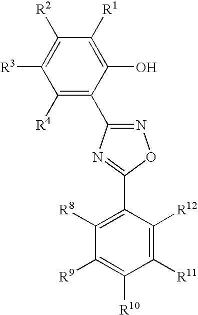

wherein: (a) R¹, R², R³ and R⁴ are as defined and provided for above; (b) R⁸, R⁹, R¹⁰, R¹¹, and R¹² are the same or different and are independently selected from the group consisting of hydrogen, alkyl, substituted alkyl, cycloalkyl, substituted cycloalkyl, heteroalkyl, substituted heteroalkyl, heterocycloalkyl, substituted heterocycloalkyl, aryl, substituted aryl, heteroaryl, substituted heteroaryl, alkoxy, aryloxy, halo, silyl, boryl, phosphino, amino, thioalkyl, thioaryl, nitro, and combinations thereof, and optionally with two or more of R⁸, R⁹, R¹⁰, R¹¹, and R¹² being joined to form a fused ring system having up to 50 atoms, not counting hydrogen atoms.

In yet another aspect, the present invention is directed to a catalyst formed from (a) a complex or a composition as set forth above, and (b) an activator, a combination of activators, or an activating technique.

In yet another aspect, the present invention is directed to one or more catalysts as detailed above, wherein the catalyst is optionally supported before or after activation.

In yet another aspect, the present invention is directed to a polymerization process comprising subjecting one or more monomers, and in particular an olefin monomer, to polymerization conditions in the presence of (a) a catalyst comprising a complex a composition as set forth above, and (b) an activator, a combination of activators, or an activating technique. In particular, the present invention is directed to such a polymerization process that is performed in solution at a temperature greater than or equal to about 120° C.

### BRIEF DESCRIPTION OF THE DRAWINGS

FIG. 1 is a crystal structure of complex M1 of the invention.

### DETAILED DESCRIPTION

The invention provides select phenol-heterocycle ligands, metal-ligand compositions or complexes formed therefrom, and their use as catalysts in a variety of polymerizations or transformations, including olefin polymerization reactions. Specifically, it has been found that phenol-oxadiazole ligands, and in particular 3-phenol-1,2,4-oxadiazoles, may be used to form metal-ligand compositions or complexes (with, for example, zirconium), which are particularly well-suited for use as catalysts in various polymerization reactions and other transformations. For example, these catalysts have been found to produce higher weight-average molecular weight (Mw) polymers (e.g., polystyrenes), which have a range of stereo-sequence distributions, at a high polymerization temperature that is not matched by conventional phenol-heterocyclic catalysts.

In view of the forgoing, it is to be noted that, as used herein, the phrase “high temperature” generally refers to a temperature greater than about 100° C. (e.g., greater than about 125° C., about 150° C., about 175° C., about 200° C., about 225° C., about 250° C., or more, the reaction temperature for example being within the range of from about 120° C. to about 250° C., or about 125° C. to about 225° C., or about 130° C. to about 170° C.).

Also as used herein, the phrase “characterized by the formula” is not intended to be limiting and is used in the same way that “comprising” is commonly used. The term “independently selected” is used herein to indicate that the groups in question—e.g., R¹, R², R³, R⁴, etc.—can be identical or different (e.g., R¹, R², R³, R⁴, etc. may all be substituted alkyls, or R¹ and R² may be a substituted alkyl and R³ may be an aryl, etc.). When two or more specific R groups appear in a formula, they can also be the same or different from each other. Use of the singular includes use of the plural and vice versa (e.g., a hexane solvent, includes hexanes). A named “R” group will generally have the structure that is recognized in the art as corresponding to R groups having that name. The terms “compound” and “complex” are generally used interchangeably in this specification, but those of skill in the art may recognize certain compounds as complexes and vice versa. For the purposes of illustration, representative certain groups are defined herein. These definitions are intended to supplement and illustrate, not preclude, the definitions known to those of skill in the art.

“Optional” or “optionally” means that the subsequently described event or circumstance may or may not occur, and that the description includes instances where the event or circumstance occurs and instances where it does not. For example, the phrase “optionally substituted hydrocarbyl” means that a hydrocarbyl moiety may or may not be substituted and that the description includes both unsubstituted hydrocarbyl and hydrocarbyl where there is substitution.

The term “substituted” as in “substituted hydrocarbyl,” “substituted aryl,” “substituted alkyl,” and the like, means that in the group in question (i.e., the hydrocarbyl, alkyl, aryl or other moiety that follows the term), at least one hydrogen atom bound to a carbon atom is replaced with one or more substituent groups such as hydroxy, alkoxy, alkylthio, phosphino, amino, halo, silyl, and the like. When the term “substituted” introduces a list of possible substituted groups, it is intended that the term apply to every member of that group. That is, the phrase “substituted alkyl, alkenyl and alkynyl” is to be interpreted as “substituted alkyl, substituted alkenyl and substituted alkynyl.” Similarly, “optionally substituted alkyl, alkenyl and alkynyl” is to be interpreted as “optionally substituted alkyl, optionally substituted alkenyl and optionally substituted alkynyl.”

The term “saturated” refers to the lack of double and triple bonds between atoms of a radical group such as ethyl, cyclohexyl, pyrrolidinyl, and the like. The term “unsaturated” refers to the presence of one or more double and triple bonds between atoms of a radical group such as vinyl, allyl, acetylide, oxazolinyl, cyclohexenyl, acetyl and the like, and specifically includes alkenyl and alkynyl groups, as well as groups in which double bonds are delocalized, as in aryl and heteroaryl groups as defined below.

The terms “cyclo” and “cyclic” are used herein to refer to saturated or unsaturated radicals containing a single ring or multiple condensed rings. Suitable cyclic moieties include, for example, cyclopentyl, cyclohexyl, cyclooctenyl, bicyclooctyl, phenyl, napthyl, pyrrolyl, furyl, thiophenyl, imidazolyl, and the like. In particular embodiments, cyclic moieties include between 3 and 200 atoms other than hydrogen, between 3 and 50 atoms other than hydrogen or between 3 and 20 atoms other than hydrogen.

The term “hydrocarbyl” refers to hydrocarbyl radicals containing 1 to about 50 carbon atoms, specifically 1 to about 24 carbon atoms, most specifically 1 to about 16 carbon atoms, including branched or unbranched, cyclic or acyclic, saturated or unsaturated species, such as alkyl groups, alkenyl groups, aryl groups, and the like.

The term “alkyl” as used herein refers to a branched or unbranched saturated hydrocarbon group typically, although not necessarily, containing 1 to about 50 carbon atoms, such as methyl, ethyl, n-propyl, isopropyl, n-butyl, isobutyl, sec-butyl, t-butyl, octyl, decyl, and the like, as well as cycloalkyl groups such as cyclopentyl, cyclohexyl and the like. Generally, although again not necessarily, alkyl groups herein may contain 1 to about 20 carbon atoms.

The term “alkenyl” as used herein refers to a branched or unbranched, cyclic or acyclic hydrocarbon group typically although not necessarily containing 2 to about 50 carbon atoms and at least one double bond, such as ethenyl, n-propenyl, isopropenyl, n-butenyl, isobutenyl, octenyl, decenyl, and the like. Generally, although again not necessarily, alkenyl groups herein contain 2 to about 20 carbon atoms.

The term “alkynyl” as used herein refers to a branched or unbranched, cyclic or acyclic hydrocarbon group typically although not necessarily containing 2 to about 50 carbon atoms and at least one triple bond, such as ethynyl, n-propynyl, isopropynyl, n-butynyl, isobutynyl, octynyl, decynyl, and the like. Generally, although again not necessarily, alkynyl groups herein may have 2 to about 20 carbon atoms.

The term “aromatic” is used in its usual sense, including unsaturation that is essentially delocalized across several bonds around a ring. The term “aryl” as used herein refers to a group containing an aromatic ring. Aryl groups herein include groups containing a single aromatic ring or multiple aromatic rings that are fused together, linked covalently, or linked to a common group such as a methylene or ethylene moiety. More specific aryl groups contain one aromatic ring or two or three fused or linked aromatic rings, e.g., phenyl, naphthyl, biphenyl, anthracenyl, or phenanthrenyl. In particular embodiments, aryl substituents include 1 to about 200 atoms other than hydrogen, typically 1 to about 50 atoms other than hydrogen, and specifically 1 to about 20 atoms other than hydrogen. In some embodiments herein, multi-ring moieties are substituents and in such embodiments the multi-ring moiety can be attached at an appropriate atom. For example, “naphthyl” can be 1-naphthyl or 2-naphthyl; “anthracenyl” can be 1-anthracenyl, 2-anthracenyl or 9-anthracenyl; and “phenanthrenyl” can be 1-phenanthrenyl, 2-phenanthrenyl, 3-phenanthrenyl, 4-phenanthrenyl or 9-phenanthrenyl.

The term “alkoxy” as used herein intends an alkyl group bound through a single, terminal ether linkage; that is, an “alkoxy” group may be represented as —O-alkyl where alkyl is as defined above. The term “aryloxy” is used in a similar fashion, and may be represented as —O-aryl, with aryl as defined below. The term “hydroxy” refers to —OH.

Similarly, the term “alkylthio” as used herein intends an alkyl group bound through a single, terminal thioether linkage; that is, an “alkylthio” group may be represented as —S-alkyl where alkyl is as defined above. The term “arylthio” is used similarly, and may be represented as —S-aryl, with aryl as defined below. The term “mercapto” refers to —SH.

The terms “halo” and “halogen” are used in the conventional sense to refer to a chloro, bromo, fluoro or iodo radical.

The terms “heterocycle” and “heterocyclic” refer to a cyclic radical, including ring-fused systems, including heteroaryl groups as defined below, in which one or more carbon atoms in a ring is replaced with a heteroatom—that is, an atom other than carbon, such as nitrogen, oxygen, sulfur, phosphorus, boron or silicon. Heterocycles and heterocyclic groups include saturated and unsaturated moieties, including heteroaryl groups as defined below. Specific examples of heterocycles include pyrrolidine, pyrroline, furan, tetrahydrofuran, thiophene, imidazole, oxazole, thiazole, indole, and the like, including any isomers of these. Additional heterocycles are described, for example, in Alan R. Katritzky, Handbook of Heterocyclic Chemistry, Pergammon Press, 1985, and in Comprehensive Heterocyclic Chemistry, A. R. Katritzky et al., Eds, Elsevier, 2d. ed., 1996. The term “metallocycle” refers to a heterocycle in which one or more of the heteroatoms in the ring or rings is a metal.

The term “heteroaryl” refers to an aryl radical that includes one or more heteroatoms in the aromatic ring. Specific heteroaryl groups include groups containing heteroaromatic rings such as thiophene, pyridine, pyrazine, isoxazole, pyrazole, pyrrole, furan, thiazole, oxazole, imidazole, isothiazole, oxadiazole, triazole, and benzo-fused analogues of these rings, such as indole, carbazole, substituted carbazoles, benzofuran, benzothiophene, benzimidiazole, benzthiazole, benzoxazoles, indazole and the like and isomers thereof, e.g., reverse isomers.

More generally, the modifiers “hetero” and “heteroatom-containing”, as in “heteroalkyl” or “heteroatom-containing hydrocarbyl group” refer to a molecule or molecular fragment in which one or more carbon atom is replaced with a heteroatom. Thus, for example, the term “heteroalkyl” refers to an alkyl substituent that is heteroatom-containing. When the term “heteroatom-containing” introduces a list of possible heteroatom-containing groups, it is intended that the term apply to every member of that group. That is, the phrase “heteroatom-containing alkyl, alkenyl and alkynyl” is to be interpreted as “heteroatom-containing alkyl, heteroatom-containing alkenyl and heteroatom-containing alkynyl.”

By “divalent” as in “divalent hydrocarbyl”, “divalent alkyl”, “divalent aryl” and the like, is meant that the hydrocarbyl, alkyl, aryl or other moiety is bonded at two points to atoms, molecules or moieties with the two bonding points being covalent bonds.

As used herein the term “silyl” refers to the —SiZ¹Z²Z³ radical, where each of Z¹, Z², and Z³ is independently selected from the group consisting of hydrogen and optionally substituted alkyl, alkenyl, alkynyl, heteroatom-containing alkyl, heteroatom-containing alkenyl, heteroatom-containing alkynyl, aryl, heteroaryl, alkoxy, aryloxy, amino, silyl and combinations thereof.

As used herein the term “boryl” refers to the —BZ¹Z² group, where each of Z¹ and Z² is as defined above. As used herein, the term “phosphino” refers to the group —PZ¹Z², where each of Z¹ and Z² is as defined above. As used herein, the term “phosphine” refers to the group —PZ¹Z²Z³, where each of Z¹, Z³ and Z² is as defined above. The term “amino” is used herein to refer to the group —NZ¹Z², where each of Z¹ and Z² is as defined above. The term “amine” is used herein to refer to the group —NZ¹Z²Z³, where each of Z¹, Z² and Z³ is as defined above.

In this specification, ligand binding is sometimes referred to as (2,1) complexation, with the first number representing the number of coordinating atoms and second number representing the number of anionic sites on the phenol-heterocycle ligand, when the metal-ligand bonding is considered from an ionic bonding model perspective, with the metal considered to be cationic and the ligand considered to be anionic. From a covalent bonding model perspective, a (2,1) complex may be considered to be a complex in which the phenol-heterocycle ligand is bound to the metal center via one covalent bond and one dative bond and examples of (2,1) complexation include the complex example labeled M1 below.

Other abbreviations used herein include: “Cbz” to refer to N-carbazole; “ⁱPr” to refer to isopropyl; “tBu” to refer to tert-butyl; “Me” to refer to methyl; “Et” to refer to ethyl; “Ph” to refer to phenyl; “Mes” to refer to mesityl (2,4,6-trimethyl phenyl); “TFA” to refer to trifluoroacetate and “THF” to refer to tetrahydrofuran.

It is to be noted that the ligands, compounds, complexes, methods, etc. of the present invention are preferably directed to, derived from, based on, comprise or utilize 3-phenol-1,2,4-oxadiazole ligands, rather than 5-phenol-1,2,4-oxadiazole ligands, as illustrated below.

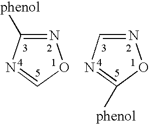

Furthermore, for one or more embodiments herein, the ligands, compounds, complexes, methods, etc., disclosed in U.S. Patent Publication No. US 2006-0135713 A1 to Leclerc et al. (the disclosure of which is incorporated by reference herein), may not specifically be within the scope of the present disclosure.

It is to be still further noted that the complexes disclosed herein can include “mono” ligand and “bis” ligand complexes. Examples of “mono” ligand complexes are those wherein a single phenol-heterocycle ligand is complexed to the metal atom. Examples of “bis” ligand complexes are those wherein two phenol-heterocycle ligands are complexed to the metal atom. It should also be understood that “bis” ligands can include two different phenol-heterocycle ligands.

In general, in one aspect, the ligands according to the present invention can be characterized broadly as monoanionic ligands having a phenol and a heterocyclic or substituted heterocyclic group. Preferred ligand substituents for some particular monomers are described in more detail below. In some embodiments, the ligands of the invention can be characterized by the following (I) or (IA):

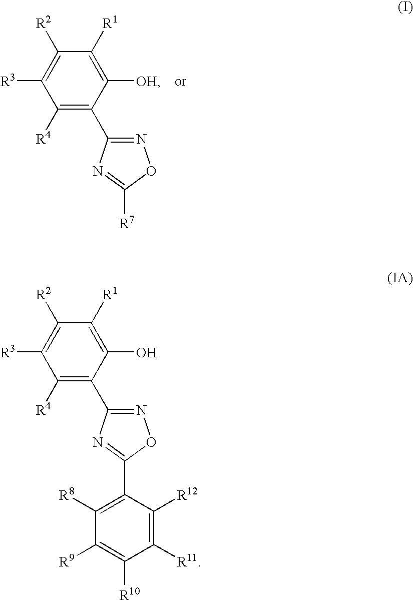

In general, R¹, R², R³, R⁴, R⁷, R⁸, R⁹, R¹⁰, R¹¹, and R¹² may be independently selected from the group consisting of hydrogen, alkyl, substituted alkyl, cycloalkyl, substituted cycloalkyl, heteroalkyl, substituted heteroalkyl, heterocycloalkyl, substituted heterocycloalkyl, aryl, substituted aryl, heteroaryl, substituted heteroaryl, alkoxy, aryloxy, halo, silyl, boryl, phosphino, amino, thioalkyl, thioaryl, nitro, and combinations thereof, with the exception that R¹ may not be hydrogen, and/or optionally with two or more of R¹, R², R³ and R⁴ being joined to form a fused ring system having up to 50 atoms, not counting hydrogen atoms, and/or optionally with two or more of R⁸, R⁹, R¹⁰, R¹¹ and R¹² groups being joined to form a fused ring system having up to 50 atoms, not counting hydrogen atoms. In one or more preferred embodiments, however, (i) at least one of R¹, R², R³ and R⁴ is selected from the group consisting of carbazolyl and substituted carbazolyl, and further (ii) at least one of R¹, R², R³ and R⁴ is selected from the group consisting of alkyl, substituted alkyl, halo, and alkoxy, and still further (iii) R⁷ is selected from the group consisting of phenyl, substituted phenyl, and anthracenyl;

In one aspect for compounds of formula (I) and the below formulas, R¹ is selected from the group consisting of alkyl (e.g., tBu), substituted alkyl, naphthyl, substituted naphthyl, carbazolyl, substituted carbazolyl, phenyl, substituted phenyl, indolyl, substituted indolyl, adamantyl, substituted adamantyl, thiophenyl, substituted thiophenyl, benzofuranyl, substituted benzofuranyl, benzothiophenyl and substituted benzothiophenyl.

In another aspect for compounds of formula (I) and the below formulas, R⁷ is selected from the group consisting of alkyl, substituted alkyl, naphthyl, substituted naphthyl, carbazolyl, substituted carbazolyl, phenyl, substituted phenyl, indolyl, substituted indolyl, adamantyl, substituted adamantyl, thiophenyl, substituted thiophenyl, benzofuranyl, substituted benzofuranyl, benzothiophenyl and substituted benzothiophenyl.

In another aspect for compounds of formula (I) and the below formulas, R³ is selected from the group consisting of alkyl (e.g., tBu), substituted alkyl, halo, alkoxy (e.g., methoxy), phenyl and substituted phenyl.

In particular, R¹ is selected from the group consisting of t-butyl, naphthyl, substituted naphthyl, carbazolyl, substituted carbazolyl, phenyl and substituted phenyl; and/or R³ is selected from the group consisting of alkyl, substituted alkyl, halo, alkoxy, phenyl and substituted phenyl; and/or R⁷ is selected from the group consisting of substituted phenyl and anthracenyl.

Ligands within the scope of one or more embodiments of the present invention may be selected, for example, from the following (which is presented for illustration and therefore should not be viewed in a limiting sense):

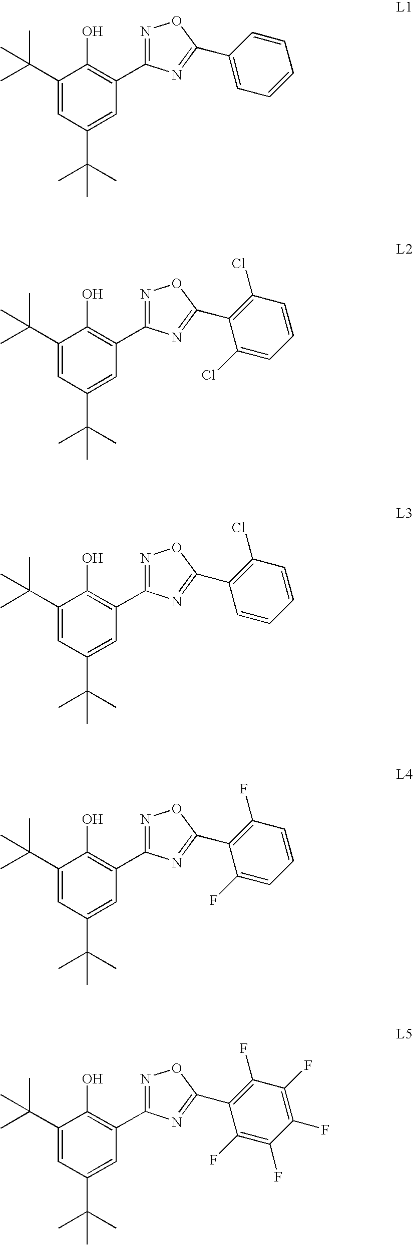

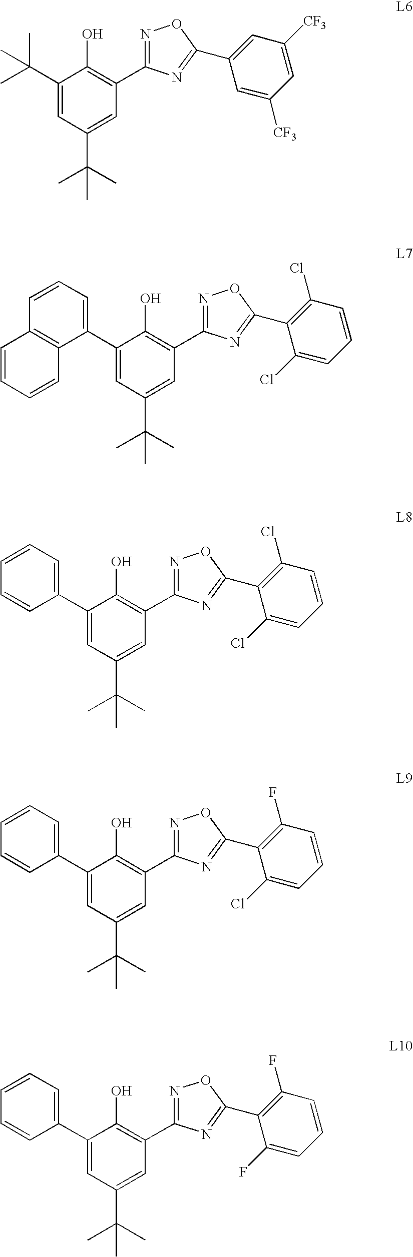

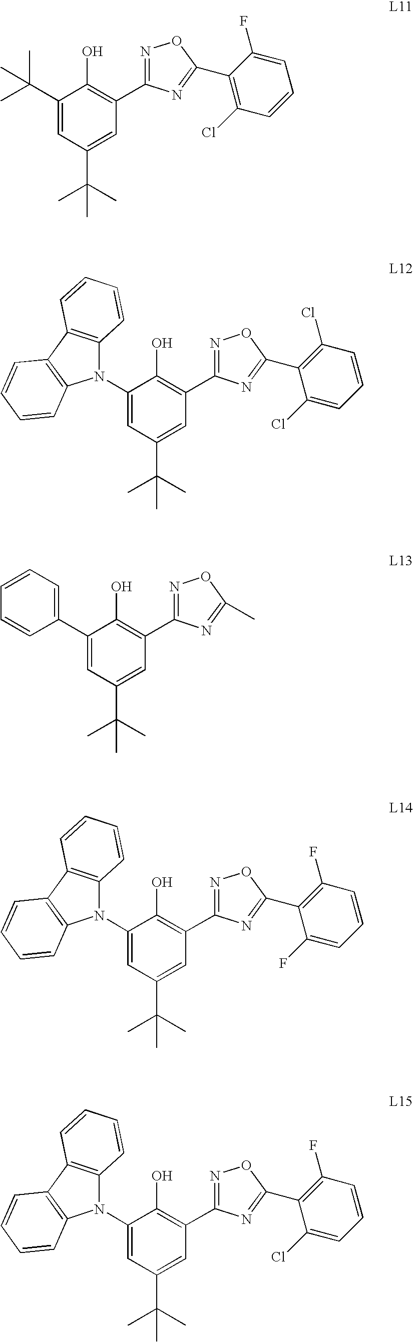

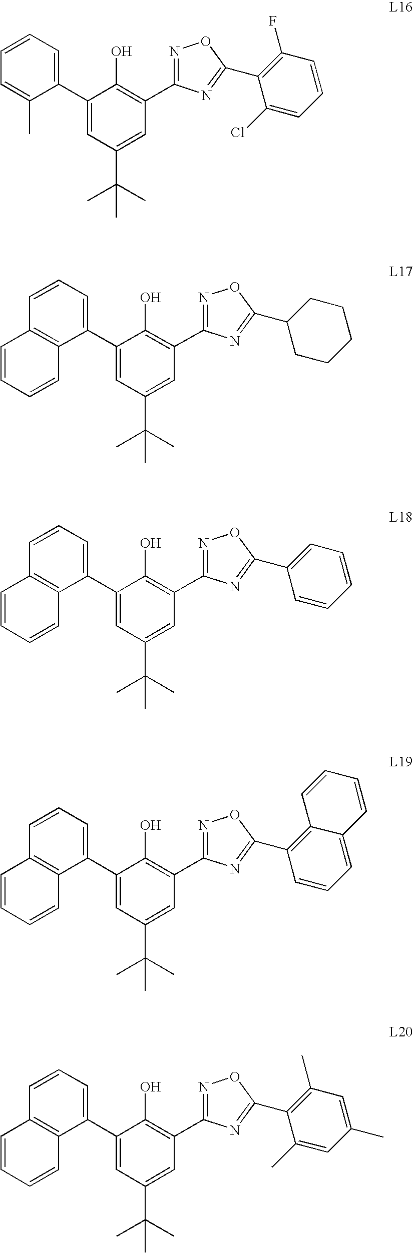

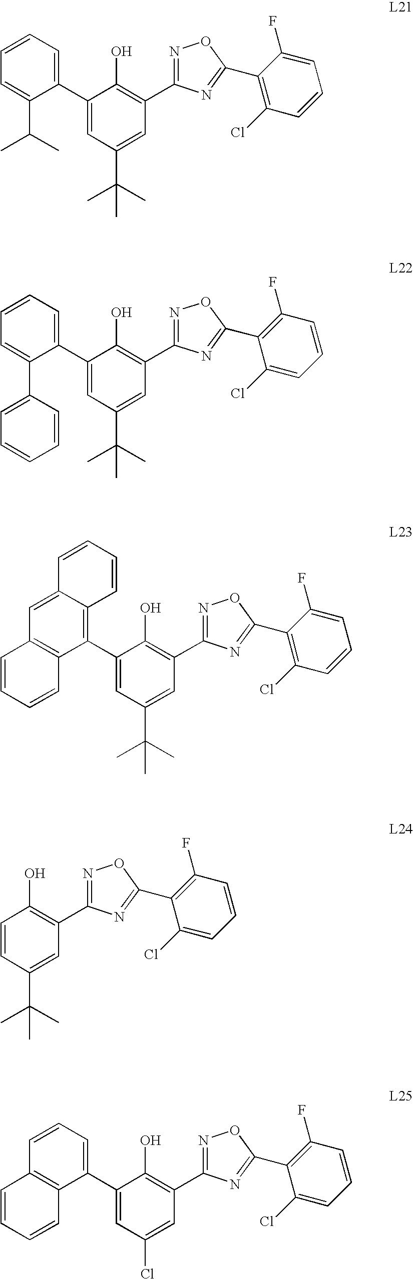

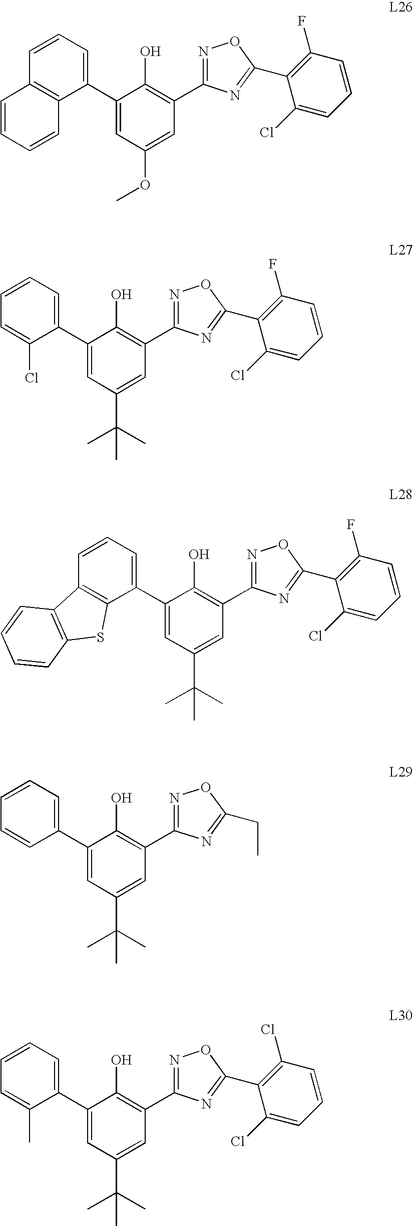

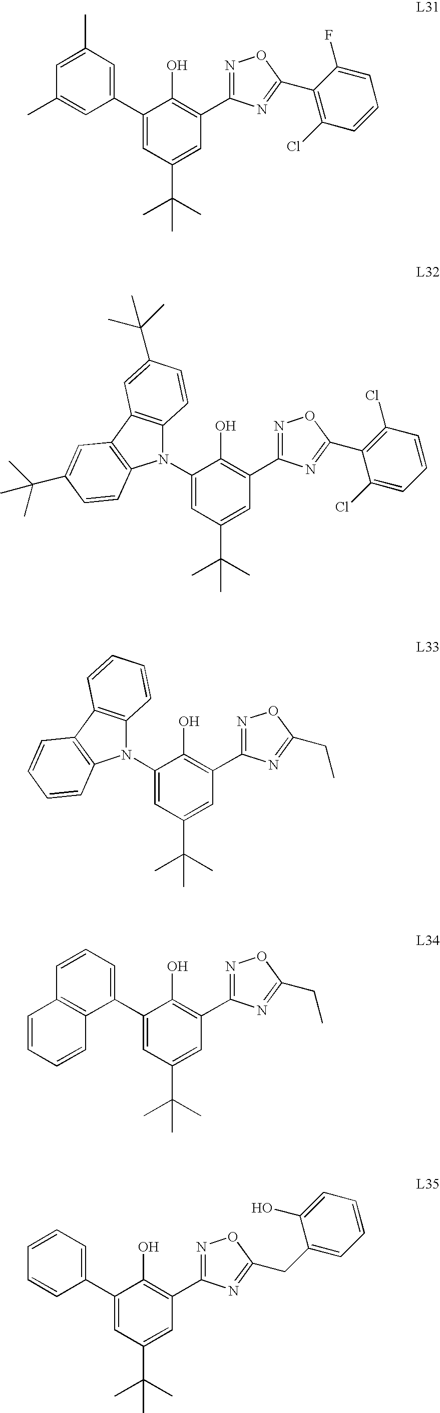

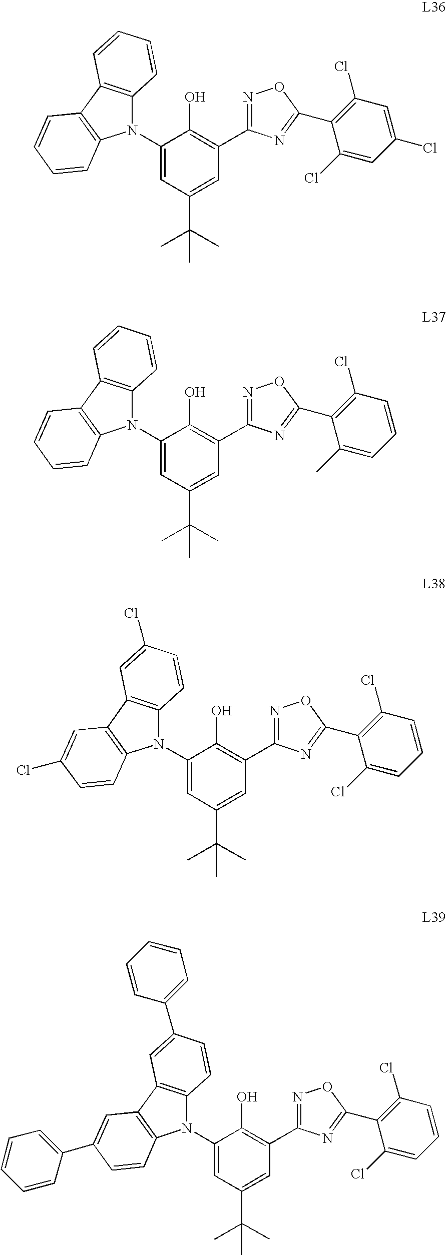

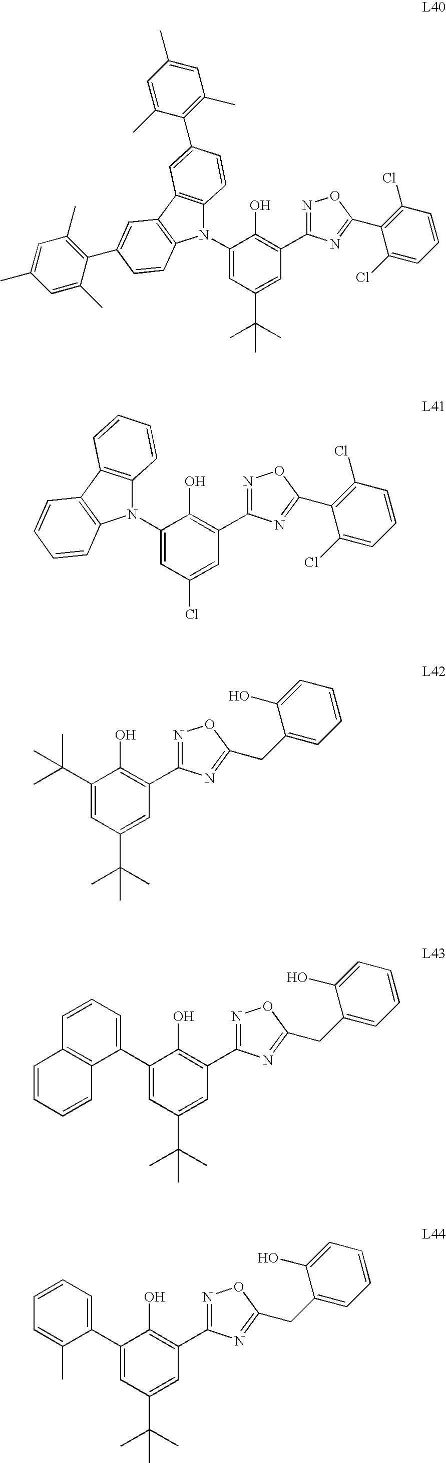

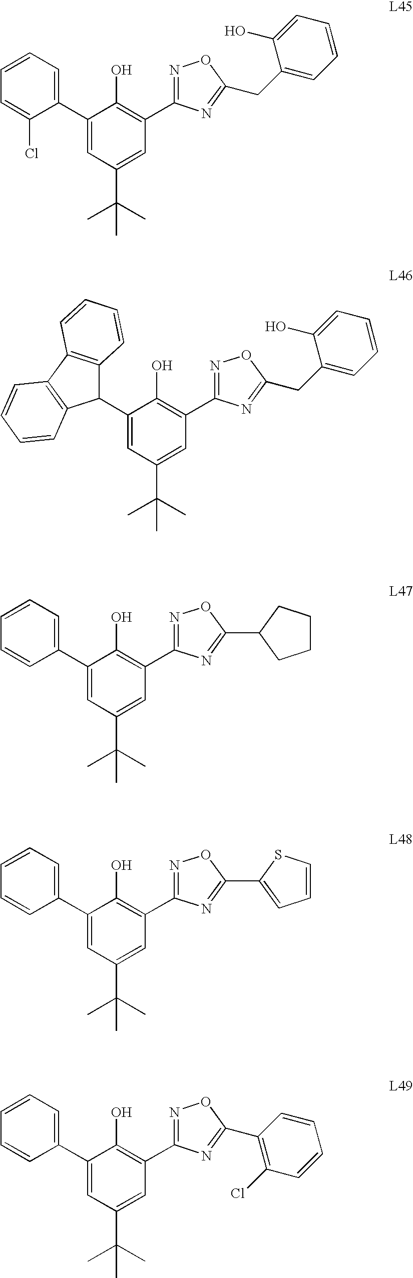

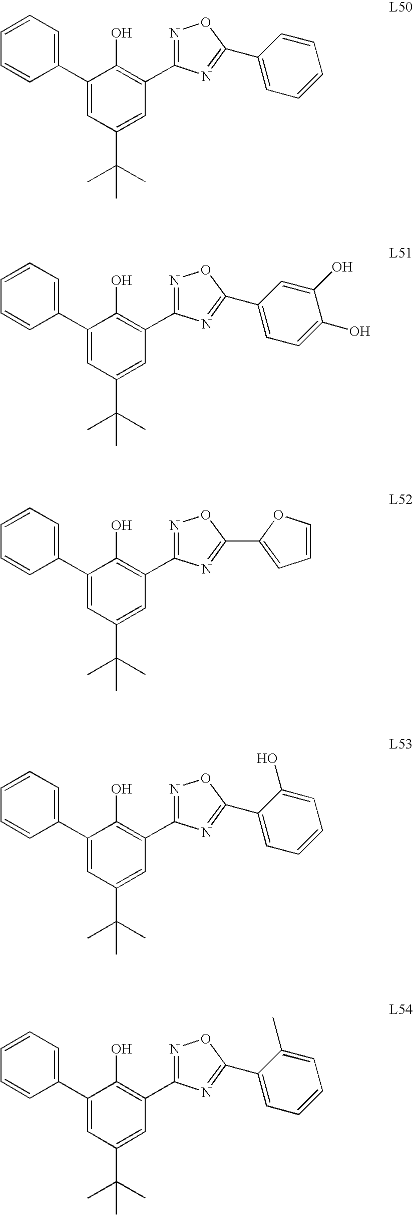

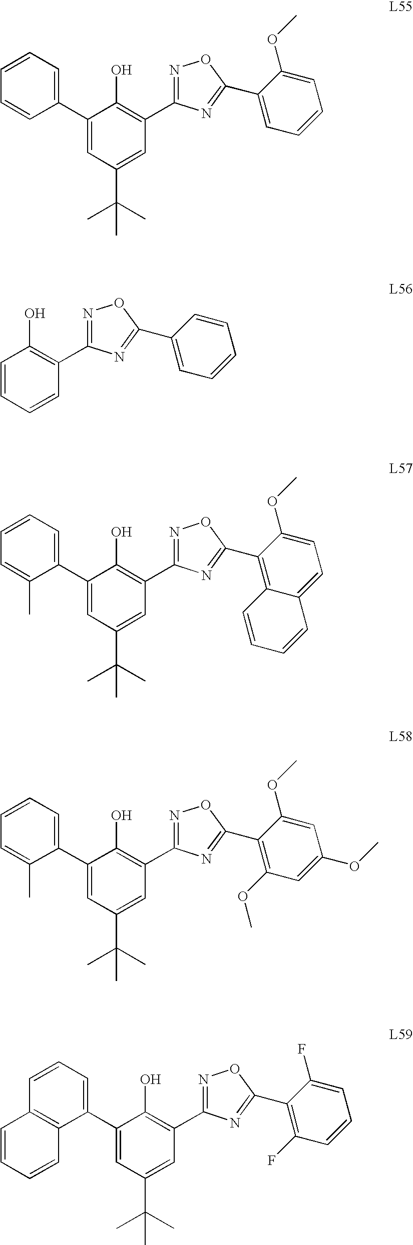

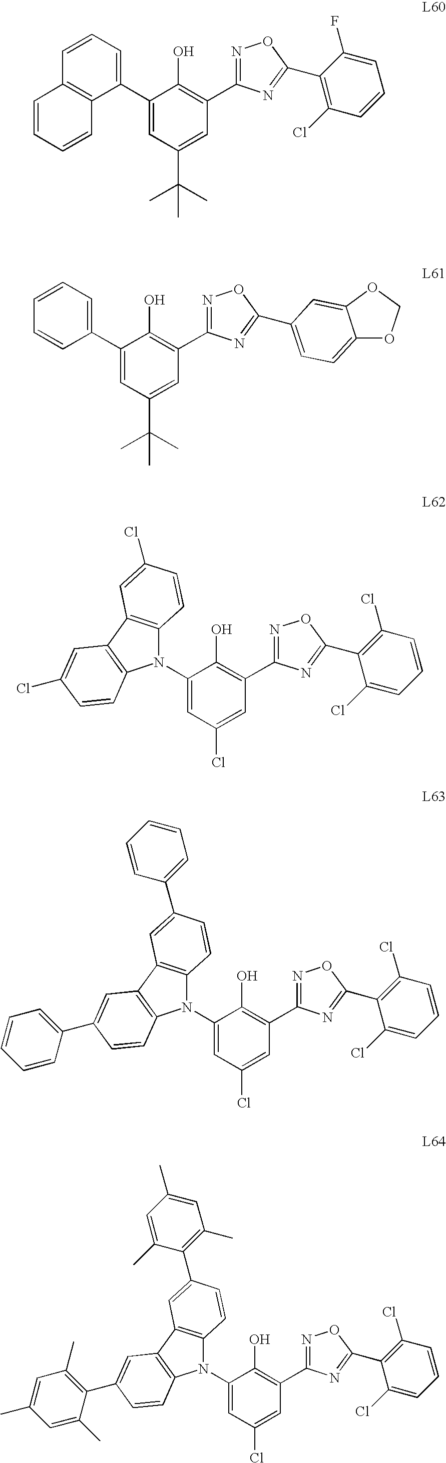

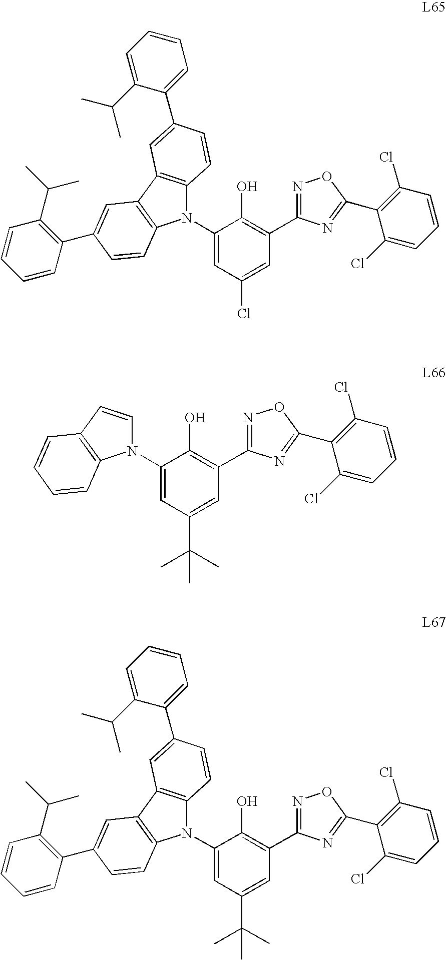

In one particular embodiment, the ligands of the present invention are selected from among L12, L14, L15, L36-41 and L62-67.

In general, in one aspect, the invention provides compositions of matter, including ligands, compositions and metal-ligand complexes, that include a compound characterized by the formula (II) or (IIA):

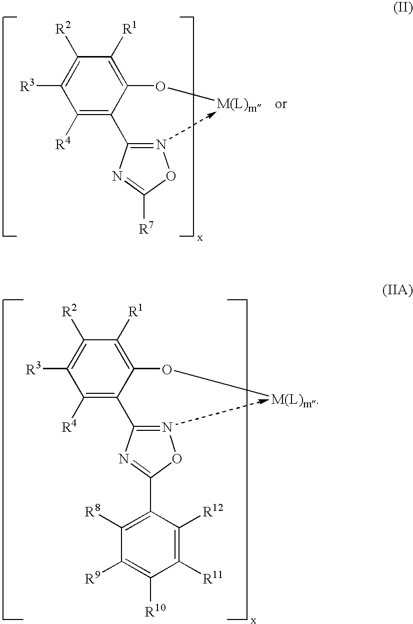

R¹, R², R³, R⁴, R⁷, R⁸, R⁹, R¹⁰, R¹¹, and R¹² are defined above. M is a metal selected from the group consisting of groups 3 through 6 of the Periodic Table of Elements and Lanthanides. Each L is independently selected from the group consisting of hydrogen, halogen, optionally substituted alkyl, heteroalkyl, allyl, diene, alkenyl, heteroalkenyl, alkynyl, heteroalkynyl, aryl, heteroaryl, alkoxy, aryloxy, boryl, silyl, amino, phosphino, ether, thioether, phosphine, amine, carboxylate, alkylthio, arylthio, 1,3-dionate, oxalate, carbonate, nitrate, sulphate, and combinations thereof; while x is 1 or 2 or 3 and m″ is 0, 1, 2, or 3. The bond between the heteroaromatic nitrogen (N) and the metal (M) is dative or absent.

Specific metal complexes of this invention include:

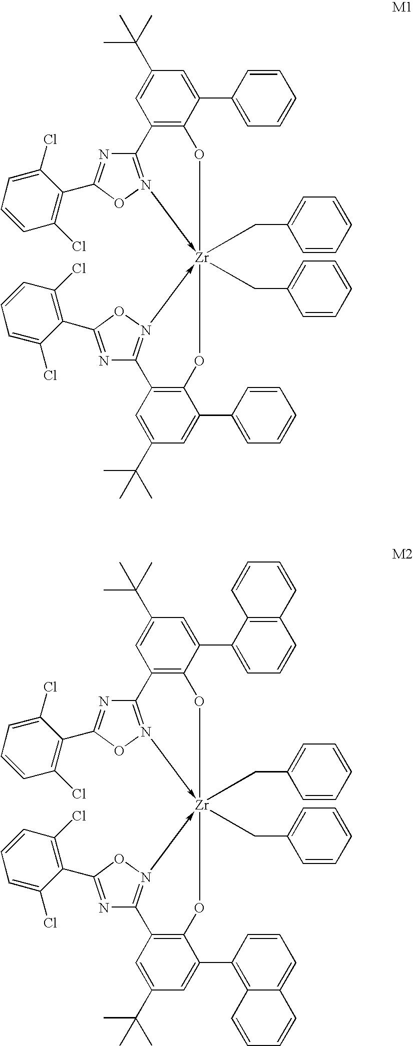

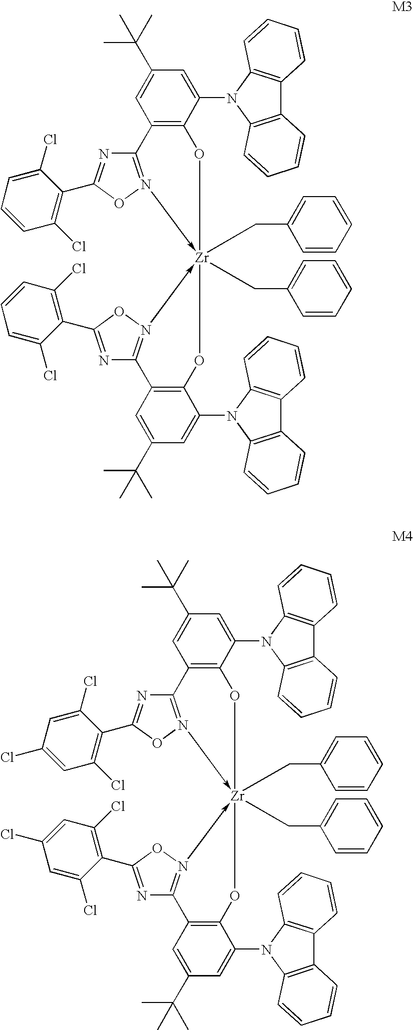

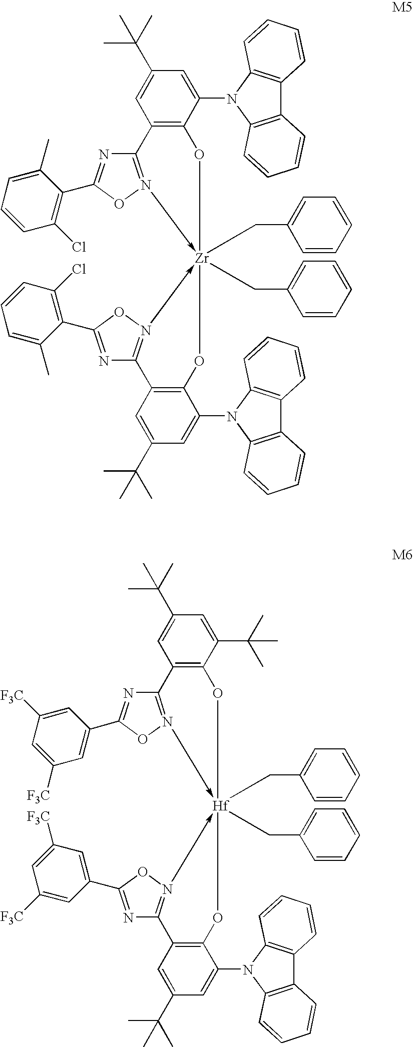

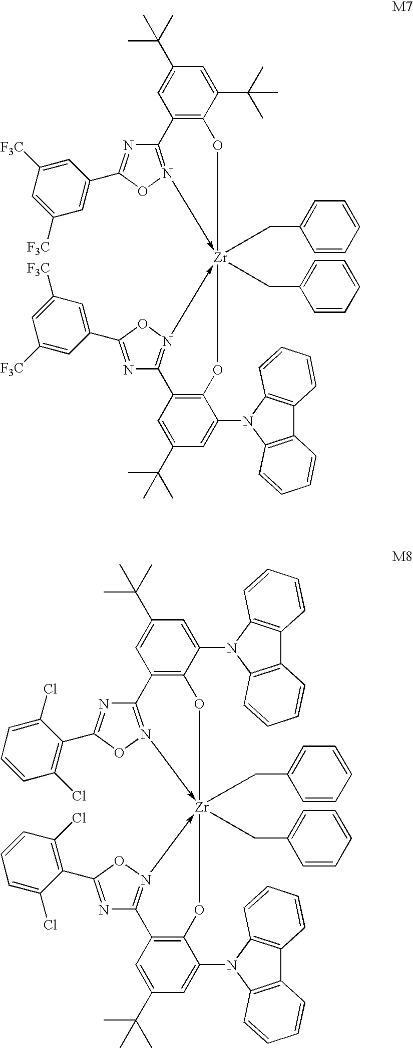

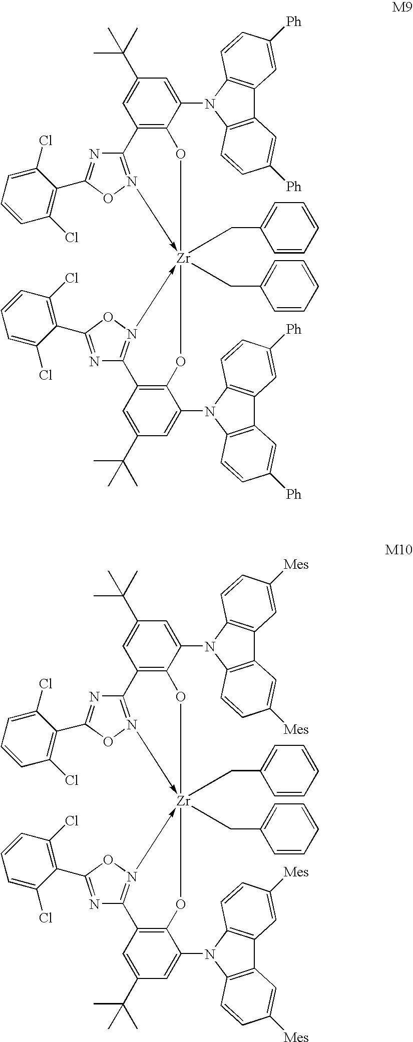

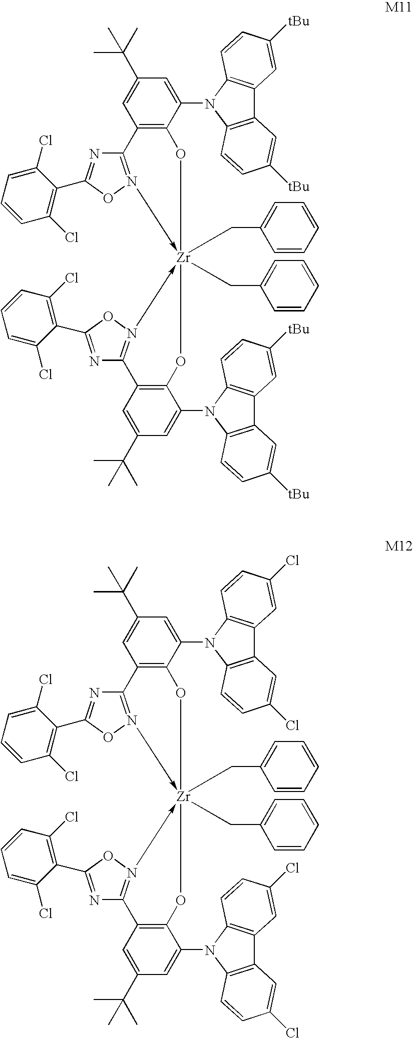

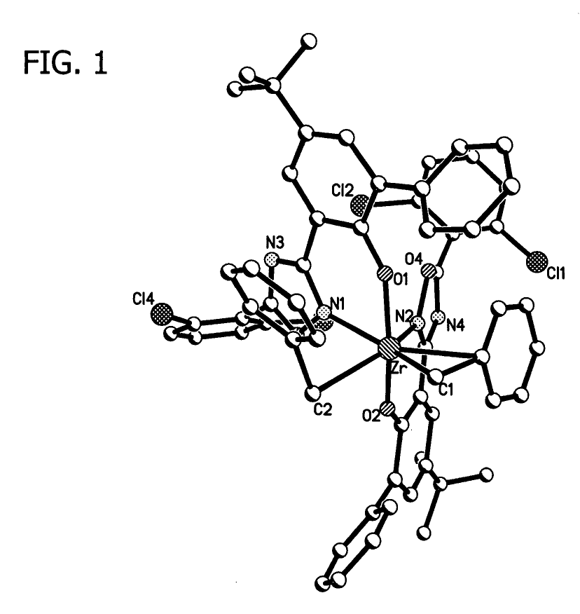

Referring now to FIG. 1, it is to be noted that a crystal structure of M1, which is further detailed elsewhere herein and which an exemplary embodiment of a metal complex according to the present invention, is illustrated therein.

In general, in still another aspect, the invention provides arrays of materials. The arrays include a substrate having at least 8 members associated with regions of the substrate. Each array member is different from the other members of the array. Each array member includes a compound, composition or complex according to one of the aspects described above.

In general, in another aspect, the invention provides catalytic methods. In the methods, one or more reagent is reacted in the presence of a catalyst comprising a composition or complex as described above, and optionally one or more activators, under conditions sufficient to yield one or more reaction products.

In general, in another aspect, the invention provides polymerization processes that employ the composition or complexes of the invention, optionally in the presence of at least one activator. In particular embodiments, the activator can include an ion forming activator and, optionally, a group 13 reagent. The activator can include an alumoxane.

In general, in another aspect, the invention provides a process for the polymerization of an α-olefin. According to the process, at least one α-olefin is polymerized in the presence of a catalyst formed from a composition or complex of the invention, optionally in the presence of one or more activators, under polymerization conditions sufficient to form a polymer, and in some instances a substantially stereoregular polymer.

In general, in another aspect, the invention provides a process for polymerizing ethylene and at least one α-olefin. According to the process, ethylene is polymerized in the presence of at least one α-olefin in the presence of a catalyst formed from a composition or complex of the invention, optionally in the presence of one or more activators.

In general, in another aspect, the invention provides a process for polymerizing at least one monomer. The process includes providing a reactor with reactor contents including at least one polymerizable monomer and a composition or complex of the invention, and subjecting the reactor contents to polymerization conditions. In particular embodiments, the at least one polymerizable monomer can include ethylene and propylene, ethylene and 1-hexene, ethylene and 1-butene, 1-octene, 1-decene, ethylene and styrene, ethylene and a cyclic alkene, ethylene and a diene, or ethylene, propylene, and a diene selected from the group consisting of ethylidenenorbornene, dicyclopentadiene, and 1,4-hexadiene.

In general, in another aspect, the invention provides a process for the polymerization of a polymerizable monomer. According to the process, a composition or complex of the invention is provided, the composition or complex is optionally activated, and at least one polymerizable monomer is polymerized in the presence of the activated composition or complex to produce a distribution of product polymers that is at least bimodal by one or more of molecular weight or composition.

The invention can be implemented to provide one or more of the following advantages. The ligands, compositions, complexes and polymerization methods of the invention can be used to provide catalysts exhibiting enhanced activity. Catalysts incorporating the ligands, compositions and/or complexes can be used to catalyze a variety of transformations, such as olefin oligomerization (specifically dimerization, trimerization and tetramerization) or polymerization. By selecting an appropriate ligand and metal, compositions and/or complexes can be obtained to provide for desired properties in the resulting product. Thus, polymers produced using the ligands, compositions, complexes, and methods of the invention can exhibit higher (or lower) melting points, higher (or lower) molecular weights, and/or higher (or lower) polydispersities, than polymers produced using prior known catalysts. In some embodiments, polymer products having bi- or multi-modal distributions of product composition and/or molecular weight can be obtained by selecting a single catalyst precursor and activating it under certain conditions. Catalysts incorporating the ligands, compositions and/or complexes can be used according to the polymerization methods of the invention to produce polymers under commercially desirable polymerization conditions. Catalysts incorporating the ligands, compositions and complexes of the invention can exhibit catalytic activity at higher temperatures than prior known catalysts. Copolymerization processes (e.g., ethylene/α-olefin copolymerizations) using the ligands, compositions and complexes of the invention can exhibit higher (or lower) comonomer incorporation than processes involving prior known catalysts. Chiral compositions and/or complexes according to the invention can be used to catalyze stereoselective, enantioselective or diastereoselective transformations.

For compounds of formula (I) or (II), preferred “R” groups include, but are not limited to, the following (various embodiments therefore including one or more of these groups, or any of the possible combinations or permutations of these groups): R¹ is selected from the group consisting of tBu, phenyl, naphthyl, N-carbazole, and 3,6-substituted N-carbazole; R² is hydrogen; R³ is selected from the group consisting of tBu, methoxy, and halo (or more specifically chloride); R⁴ is hydrogen; R⁷ is aryl (or more specifically phenyl); and, when present: R⁸ is selected from the group consisting of chloride, fluoride, and methyl; R⁹ and R¹¹ are independently selected from the group consisting of hydrogen and CF₃; R¹⁰ is selected from the group consisting of hydrogen and chloride; and/or R¹² is selected from the group consisting of chloride, fluoride, and methyl. Among these substituent options, preferred combinations include, but are not limited to: R¹=carbazole, R³=halo (e.g., chloro), and R⁷=dihalophenyl (e.g., 2,6-dichlorophenyl); or, R¹=3,6-diphenylcarbazole, R³=tBu, and R⁷=dihalophenyl (e.g., 2,6-dichlorophenyl).

The choice of particular heterocyclic ligand can have a strong influence on the catalysis of particular transformations. Thus, the choice of substituent in the ligands of the invention when incorporated in a polymerization catalyst can affect catalyst activity, thermal stability, molecular weight and molecular weight distribution of the product polymer, or the degree and/or kind of stereo- or regioerrors, as well as other factors known to be significant in the production of various polymers. For example, as shown below in Tables 3A and 3B, by selecting particular heterocyclic ligands, the molecular weight and activity of the resulting catalyst are increased; specifically, by choosing the L12 ligand, the molecular weight more than doubled when compared to catalyst compounds having the L2, L7, and/or L8 ligands; the L14 ligand produced a compound with greater molecular weight and activity as compared to the L10-ligand containing compound; and the L15 ligand produced similar results when compared with the L9 ligand. Similarly, in Table 3B, compounds containing the L41 and L40 ligands were higher in molecular weight, and the compound containing L41 had an increased activity as compared to the compound with the L12 ligand.

The ligands of the invention can be prepared using known procedures, such as those described, for example, in March, Advanced Organic Chemistry, Wiley, New York 1992 (4^{th} Ed.), and in Katritzky et al., Comprehensive Heterocyclic Chemistry, Elsevier, N.Y. 1984 (1^{st} Ed.) & 1996 (2^{nd} Ed.). Specifically, in some embodiments the ligands of the invention can be prepared according to the general procedures that follow.

Once the desired ligand is formed, it can be combined with a metal atom, ion, compound or other metal precursor compound, and in some embodiments the present invention encompasses compositions that include any of the above-mentioned ligands in combination with an appropriate metal precursor and an optional activator. For example, in some embodiments, the metal precursor can be an activated metal precursor, which refers to a metal precursor (described below) that has been combined or reacted with an activator (described below) prior to combination or reaction with the ancillary ligand. As noted above, in one aspect the invention provides compositions that include such combinations of ligand and metal atom, ion, compound or precursor. In some applications, the ligands are combined with a metal compound or precursor and the product of such combination is not determined, if a product forms. For example, the ligand may be added to a reaction vessel at the same time as the metal or metal precursor compound along with the reactants, activators, scavengers, etc. Additionally, the ligand can be modified prior to addition to or after the addition of the metal precursor, e.g. through a deprotonation reaction or some other modification.

In general, the metal precursor compounds can be characterized by the general formula M(L)ₘ where M is a metal selected from the group consisting of groups 3-6 and lanthanides of the periodic table of elements and m is 1, 2, 3, 4, 5, or 6. Thus, in particular embodiments M can be selected from scandium, yttrium, titanium, zirconium, hafnium, vanadium, niobium, tantalum, chromium, molybdenum, tungsten, lanthanum, cerium, praseodymium, neodymium, samarium, europium, gadolinium, terbium, dysprosium, holmium, erbium, thulium, ytterbium, and lutetium. Each L is a ligand independently selected from the group consisting of hydrogen, halogen, optionally substituted alkyl, heteroalkyl, allyl, diene, alkenyl, heteroalkenyl, alkynyl, heteroalkynyl, aryl, heteroaryl, alkoxy, aryloxy, boryl, silyl, amino, phosphino, ether, thioether, phosphine, amine, carboxylate, alkylthio, arylthio, 1,3-dionate, oxalate, carbonate, nitrate, sulphate, and combinations thereof. Optionally, two or more L groups are joined into a ring structure. One or more of the ligands L may be ionically bonded to the metal M and, for example, L may be a non-coordinated or loosely coordinated or weakly coordinated anion (e.g., L may be selected from the group consisting of those anions described below in the conjunction with the activators). (See Marks et al., Chem. Rev. 2000, 100, 1391-1434, for a detailed discussion of these weak interactions.) The metal precursors may be monomeric, dimeric or higher orders thereof. In particular embodiments, the metal precursor includes a metal selected from Ti, Zr, or Hf. In more specific embodiments, the metal precursor includes a metal selected from Zr and Hf.

Specific examples of suitable titanium, hafnium and zirconium precursors include, but are not limited to TiCl₄, Ti(CH₂Ph)₄, Ti(CH₂CMe₃)₄, Ti(CH₂SiMe₃)₄, Ti(CH₂Ph)₃Cl, Ti(CH₂CMe₃)₃Cl, Ti(CH₂SiMe₃)₃Cl, Ti(CH₂Ph)₂Cl₂, Ti(CH₂CMe₃)₂Cl₂, Ti(CH₂SiMe₃)₂Cl₂, Ti(NMe₂)₄, Ti(NEt₂)₄, Ti(O—ⁱPr)₄, and Ti(N(SiMe₃)₂)₂Cl₂, HfCl₄, Hf(CH₂Ph)₄, Hf(CH₂CMe₃)₄, Hf(CH₂SiMe₃)₄, Hf(CH₂Ph)₃Cl, Hf(CH₂CMe₃)₃Cl, Hf(CH₂SiMe₃)₃Cl, Hf(CH₂Ph)₂Cl₂, Hf(CH₂CMe₃)₂Cl₂, Hf(CH₂SiMe₃)₂Cl₂, Hf(NMe₂)₄, Hf(NEt₂)₄, and Hf(N(SiMe₃)₂)₂Cl₂, Hf(N(SiMe₃)CH₂CH₂CH₂N(SiMe₃))Cl₂, Hf(N(Ph)CH₂CH₂CH₂N(Ph))Cl₂, ZrCl₄, Zr(CH₂Ph)₄, Zr(CH₂CMe₃)₄, Zr(CH₂SiMe₃)₄, Zr(CH₂Ph)₃Cl, Zr(CH₂CMe₃)₃Cl, Zr(CH₂SiMe₃)₃Cl, Zr(CH₂Ph)₂Cl₂, Zr(CH₂CMe₃)₂Cl₂, Zr(CH₂SiMe₃)₂Cl₂, Zr(NMe₂)₄, Zr(NEt₂)₄, Zr(NMe₂)₂Cl₂, Zr(NEt₂)₂Cl₂, Zr(N(SiMe₃)₂)₂Cl₂, Zr(N(SiMe₃)CH₂ CH₂CH₂N(SiMe₃))Cl₂, and Zr(N(Ph)CH₂CH₂CH₂N(Ph))Cl₂. Lewis base adducts of these examples are also suitable as metal precursors, for example, ethers, amines, thioethers, phosphines and the like are suitable as Lewis bases. Specific examples include HfCl₄(THF)₂, HfCl₄(SMe₂)₂ and Hf(CH₂Ph)₂Cl₂(OEt₂). Activated metal precursors may be ionic or zwitterionic compounds, such as [M(CH₂Ph)₃⁺][B(C₆F₅)₄⁻] or [M(CH₂Ph)₃⁺][PhCH₂B(C₆F₅)₃⁻] where M is Zr or Hf. Activated metal precursors or such ionic compounds can be prepared in the manner shown in Pellecchia et al., Organometallics 1994, 13, 298-302; Pellecchia et al., J. Am. Chem. Soc. 1993, 115, 1160-1162; Pellecchia et al., Organometallics 1993, 13, 3773-3775 and Bochmann et al., Organometallics 1993, 12, 633-640, each of which is incorporated herein by reference.

The ligand to metal precursor compound ratio is typically in the range of about 0.01:1 to about 100:1, more specifically in the range of about 0.1:1 to about 10:1, and even more specifically about 1:1, 2:1 or 3:1.

As noted above, in another aspect, this invention relates to compositions of one or more ligands and a metal precursor compound, where the compositions include two equivalents of ligand to metal precursor compound, referred to herein as the bis-ligand embodiment. In other aspects, there is also a bis-ligand complex embodiment, specifically, those embodiments where x is 2 in the below complexation formulae. The bis-ligand composition embodiment can include two equivalents of the same phenol-heterocycle ligand or one equivalent of a first phenol-heterocycle ligand and one equivalent of a second phenol-heterocycle ligand, wherein the first and second phenol-heterocycle ligands are different from each other. In other embodiments, the ratio of the first phenol-heterocycle ligand to the second phenol-heterocycle ligand is not one to one, but rather ranges from about 1 part to 99 parts to about 99 parts to 1 part.

As also noted above, in another aspect the invention relates to metal-ligand complexes. Generally, the ligand (or optionally a modified ligand as discussed above) is mixed with a suitable metal precursor (and optionally other components, such as activators) prior to or simultaneously with allowing the mixture to be contacted with the reactants (e.g., monomers). When the ligand is mixed with the metal precursor compound, a metal-ligand complex may be formed, which may itself be an active catalyst or may be transformed into a catalyst upon activation.

The ligands, complexes or catalysts may be supported on organic or inorganic supports. Suitable supports include silicas, aluminas, clays, zeolites, magnesium chloride, polystyrenes, substituted polystyrenes and the like. Polymeric supports may be cross-linked or not. Similarly, the ligands, complexes or catalysts may be supported on supports known to those of skill in the art. See for example, Severn et al. Chem. Rev. 2005, 105, 4073-4147, particularly pages 4115-4117; Hlalky, Chem. Rev. 2000, 100, 1347-1376 and Fink et al., Chem. Rev. 2000, 100, 1377-1390, each of which is incorporated herein by reference, including the references cited therein. The compositions, complexes and/or catalysts may be contacted with an activator (described below) before or after contact with the support; alternatively, the support may be contacted with the activator prior to contact with the composition, complex or catalyst. In addition, the catalysts of this invention may be combined with other catalysts in a single reactor and/or employed in a series of reactors (parallel or serial) in order to form blends of polymer products.

It is to be noted that there is also a bis-ligand complex embodiment of this invention. Accordingly, in one aspect of the present invention, x is 2 in any of formulae (II) or (IIA).

The metal-ligand complexes and compositions described herein are active catalysts typically in combination with a suitable activator, combination of activators and or activating technique, although some of the metal-ligand complexes may be active without an activator or activating technique depending on the metal-ligand complex and on the process being catalyzed. Broadly, the activator(s) may comprise alumoxanes, Lewis acids, Bronsted acids, compatible non-interfering activators and combinations of the foregoing. These types of activators have been taught for use with different compositions or metal complexes in the following references, which are hereby incorporated by reference in their entirety: U.S. Pat. Nos. 5,599,761, 5,616,664, 5,453,410, 5,153,157, 5,064,802, EP A-277,004 and Marks et al., Chem. Rev. 2000, 100, 1391-1434. In some embodiments, ionic or ion forming activators are preferred. In other embodiments, alumoxane activators are preferred.

Suitable ion forming compounds useful as an activator in one embodiment comprise a cation that is a Bronsted acid capable of donating a proton, and an inert, compatible, non-interfering, anion, A⁻. Suitable anions include, but are not limited to, those containing a single coordination complex comprising a charge-bearing metal or metalloid core. Mechanistically, the anion should be sufficiently labile to be displaced by olefinic, diolefinic and unsaturated compounds or other neutral Lewis bases such as ethers or nitriles. Suitable metals include, but are not limited to, aluminum, gold and platinum. Suitable metalloids include, but are not limited to, boron, phosphorus, and silicon. Compounds containing anions that comprise coordination complexes containing a single metal or metalloid atom are well known and many, particularly such compounds containing a single boron atom in the anion portion, are available commercially.

Specifically, such activators may be represented by the following general formula:

(L*-H)_{d}⁺(A^{d-})

wherein L* is a neutral Lewis base; (L*-H)_{d}⁺ is a Bronsted acid; A^{d-} is a non-interfering, compatible anion having a charge of d−, and d is an integer from 1 to 3. More specifically A^{d-} corresponds to the formula: (M′³⁺Qₕ)^{d-} wherein h is an integer from 4 to 6; h−3=d; M′ is an element selected from group 13 of the periodic table; and Q is independently selected from the group consisting of hydrogen, dialkylamido, halogen, alkoxy, aryloxy, hydrocarbyl, and substituted-hydrocarbyl radicals (including halogen substituted hydrocarbyl, such as perhalogenated hydrocarbyl radicals), said Q having up to 20 carbons. In a more specific embodiment, d is one, i.e., the counter ion has a single negative charge and corresponds to the formula A⁻.

Activators comprising boron or aluminum can be represented by the following general formula:

(L*-H)⁺(M″Q₄)⁻

wherein: L* is as previously defined; M″ is boron or aluminum; and Q is a fluorinated C₁₋₂₀ hydrocarbyl group. Most specifically, Q is independently selected from the group consisting of fluorinated aryl group, such as a pentafluorophenyl group (i.e., a C₆F₅ group) or a 3,5-bis(CF₃)₂C₆H₃ group. Illustrative, but not limiting, examples of boron compounds which may be used as an activating cocatalyst in the preparation of the improved catalysts of this invention are tri-substituted ammonium salts such as: trimethylammonium tetraphenylborate, triethylammonium tetraphenylborate, tripropylammonium tetraphenylborate, tri(n-butyl)ammonium tetraphenylborate, tri(t-butyl)ammonium tetraphenylborate, N,N-dimethylanilinium tetraphenylborate, N,N-diethylanilinium tetraphenylborate, N,N-dimethylanilinium tetra-(3,5-bis(trifluoromethyl)phenyl)borate, N,N-dimethyl-(2,4,6-trimethylanilinium) tetraphenylborate, trimethylammonium tetrakis(pentafluorophenyl) borate, triethylammonium tetrakis(pentafluorophenyl) borate, tripropylammonium tetrakis(pentafluorophenyl) borate, tri(n-butyl)ammonium tetrakis(pentafluorophenyl) borate, tri(secbutyl)ammonium tetrakis(pentafluorophenyl) borate, N,N-dimethylanilinium tetrakis(pentafluorophenyl) borate, N,N-diethylanilinium tetrakis(pentafluorophenyl) borate, N,N-dimethyl-(2,4,6-trimethylanilinium) tetrakis(pentafluorophenyl) borate, trimethylammonium tetrakis-(2,3,4,6-tetrafluorophenylborate and N,N-dimethylanilinium tetrakis-(2,3,4,6-tetrafluorophenyl) borate; dialkyl ammonium salts such as: di-(i-propyl)ammonium tetrakis(pentafluorophenyl) borate, and dicyclohexylammonium tetrakis(pentafluorophenyl) borate; and tri-substituted phosphonium salts such as: triphenylphospnonium tetrakis(pentafluorophenyl) borate, tri(o-tolyl)phosphonium tetrakis(pentafluorophenyl) borate, and tri(2,6-dimethylphenyl)phosphonium tetrakis(pentafluorophenyl) borate, N,N-dimethylanilinium tetrakis(3,5-bis(trifluoromethyl)phenyl)borate, HNMe(C₁₈H₃₇)₂⁺B(C₆F₅)₄⁻, HNPh(C₁₈H₃₇)₂⁺B(C₆F₅)₄⁻ and ((4-nBu-Ph)NH(n-hexyl)₂)⁺B(C₆F₅)₄⁻ and ((4-nBu-Ph)NH(n-decyl)₂)⁺B(C₆F₅)₄⁻. Specific (L*-H)⁺ cations are N,N-dialkylanilinium cations, such as HNMe₂Ph⁺, substituted N,N-dialkylanilinium cations, such as (4-nBu-C₆H₄)NH(n-C₆H₁₃)₂⁺ and (4-nBu-C₆H₄)NH(n-C₁₀H₂₁)₂⁺ and HNMe(C₁₈H₃₇)₂⁺. Specific examples of anions are tetrakis(3,5-bis(trifluoromethyl)phenyl)borate and tetrakis(pentafluorophenyl)borate. In some embodiments, the specific activator is PhNMe₂H⁺ B(C₆F₅)₄—.

Other suitable ion forming activators comprise a salt of a cationic oxidizing agent and a non-interfering, compatible anion represented by the formula:

(Ox^{e+})_{d}(A^{d-})ₑ

wherein: Ox^{e+} is a cationic oxidizing agent having a charge of e+; e is an integer from 1 to 3; and A^{d-}, and d are as previously defined. Examples of cationic oxidizing agents include: ferrocenium, hydrocarbyl-substituted ferrocenium, Ag⁺, or Pb⁺². Specific embodiments of A^{d-} are those anions previously defined with respect to the Bronsted acid containing activating cocatalysts, especially tetrakis(pentafluorophenyl)borate.

Another suitable ion forming, activating cocatalyst comprises a compound that is a salt of a carbenium ion or silyl cation and a non-interfering, compatible anion represented by the formula:

©⁺A⁻

wherein: ©⁺ is a C₁₋₁₀₀ carbenium ion or silyl cation; and A⁻ is as previously defined. A preferred carbenium ion is the trityl cation, i.e. triphenylcarbenium. The silyl cation may be characterized by the formula Z⁴Z⁵Z⁶Si⁺ cation, where each of Z⁴, Z⁵, and Z⁶ is independently selected from the group consisting of hydrogen, halogen, and optionally substituted alkyl, alkenyl, alkynyl, heteroalkyl, heteroalkenyl, heteroalkynyl, aryl, heteroaryl, alkoxyl, aryloxyl, silyl, boryl, phosphino, amino, mercapto, alkylthio, arylthio, and combinations thereof. In some embodiments, a specified activator is Ph₃C⁺B(C₆F₅)₄⁻.

Other suitable activating cocatalysts comprise a compound that is a salt, which is represented by the formula (A*^{+a})_{b}(Z*J*ⱼ)^{-c}_{d} wherein A* is a cation of charge +a; Z* is an anion group of from 1 to 50, specifically 1 to 30 atoms, not counting hydrogen atoms, further containing two or more Lewis base sites; J* independently of each occurrence is a Lewis acid coordinated to at least one Lewis base site of Z*, and optionally two or more such J* groups may be joined together in a moiety having multiple Lewis acidic functionality; j is a number from 2 to 12; and a, b, c, and d are integers from 1 to 3, with the proviso that a×b is equal to c×d. See WO 99/42467, which is incorporated herein by reference. In other embodiments, the anion portion of these activating cocatalysts may be characterized by the formula ((C₆F₅)₃M″″-LN-M″″(C₆F₅)₃)⁻ where M″″ is boron or aluminum and LN is a linking group, which is specifically selected from the group consisting of cyanide, azide, dicyanamide and imidazolide. The cation portion is specifically a quaternary amine. See, e.g., LaPointe, et al., J. Am. Chem. Soc. 2000, 122, 9560-9561, which is incorporated herein by reference.

In addition, suitable activators include Lewis acids, such as those selected from the group consisting of tris(aryl)boranes, tris(substituted aryl)boranes, tris(aryl)alanes, tris(substituted aryl)alanes, including activators such as tris(pentafluorophenyl)borane. Other useful ion forming Lewis acids include those having two or more Lewis acidic sites, such as those described in WO 99/06413 or Piers, et al., J. Am. Chem. Soc., 1999, 121, 3244-3245, both of which are incorporated herein by reference. Other useful Lewis acids will be evident to those of skill in the art. In general, the group of Lewis acid activators is within the group of ion forming activators (although exceptions to this general rule can be found) and the group tends to exclude the group 13 reagents listed below. Combinations of ion forming activators may be used.

Other general activators or compounds useful in a polymerization reaction may be used. These compounds may be activators in some contexts, but may also serve other functions in the polymerization system, such as alkylating a metal center or scavenging impurities. These compounds are within the general definition of “activator,” but are not considered herein to be ion-forming activators. These compounds include a group 13 reagent that may be characterized by the formula G¹³R⁵⁰₃₋ₚDₚ where G¹³ is selected from the group consisting of B, Al, Ga, In and combinations thereof, p is 0, 1 or 2, each R⁵⁰ is independently selected from the group consisting of hydrogen, halogen, and optionally substituted alkyl, alkenyl, alkynyl, heteroalkyl, heteroalkenyl, heteroalkynyl, aryl, heteroaryl, and combinations thereof, and each D is independently selected from the group consisting of halogen, hydrogen, alkoxy, aryloxy, amino, mercapto, alkylthio, arylthio, phosphino and combinations thereof. In other embodiments, the group 13 activator is an oligomeric or polymeric alumoxane compound, such as methylalumoxane and the known modifications thereof. See, for example, Barron, “Alkylalumoxanes, Synthesis, Structure and Reactivity”, pp. 33-67 in Metallocene-Based Polyolefins: Preparation, Properties and Technology, J. Schiers and W. Kaminsky (eds.), Wiley Series in Polymer Science, John Wiley & Sons Ltd., Chichester, England, 2000, and references cited therein. In other embodiments, a divalent metal reagent may be used that is defined by the general formula M′R⁵⁰_{2-p′}D_{p′} and p′ is 0 or 1 in this embodiment and R⁵⁰ and D are as defined above. M′ is the metal and is selected from the group consisting of Mg, Ca, Sr, Ba, Zn, Cd and combinations thereof. In still other embodiments, an alkali metal reagent may be used that is defined by the general formula M^{iv}R⁵⁰ and in this embodiment R⁵⁰ is as defined above. M^{iv} is the alkali metal and is selected from the group consisting of Li, Na, K, Rb, Cs and combinations thereof. Additionally, hydrogen and/or silanes may be used in the catalytic composition or added to the polymerization system. Silanes may be characterized by the formula SiR⁵⁰_{4-q}D_{q}, where R⁵⁰ is defined as above, q is 1, 2, 3 or 4 and D is as defined above, with the proviso that there is at least one D that is a hydrogen.

The molar ratio of metal:activator (whether a composition or complex is employed as a catalyst) employed specifically ranges from 1:10,000 to 100:1, more specifically from 1:5000 to 10:1, most specifically from 1:10 to 1:1. In one embodiment of the invention, mixtures of the above compounds are used, particularly a combination of a group 13 reagent and an ion-forming activator. The molar ratio of group 13 reagent to ion-forming activator is specifically from 1:10,000 to 1000:1, more specifically from 1:5000 to 100:1, most specifically from 1:100 to 10:1. In another embodiment, the ion forming activators are combined with a group 13 reagent. Another embodiment is a combination of the above compounds having about 1 equivalent of an optionally substituted N,N-dialkylanilinium tetrakis(pentafluorophenyl) borate, and 5-30 equivalents of a group 13 reagent. In some embodiments from about 30 to 2000 equivalents of an oligomeric or polymeric alumoxane activator, such as a modified alumoxane (e.g., alkylalumoxane), can be used.

In some embodiments, the ligand or bis-ligand combination will be mixed with a suitable metal precursor compound prior to or simultaneous with allowing the mixture to be contacted to the reactants. When the ligand is mixed with the metal precursor compound, a metal-ligand complex may be formed, which may be a catalyst. Also, the ligand or ligand composition can be combined with an activated metal precursor, as described herein. In other aspects, the catalysts of the invention may be combined with other catalysts to make bi-modal polymer products; and the catalysts may be combined together in solution and/or on a solid support.

The ligands, compositions, complexes and/or catalysts of the invention can be used to catalyze a variety of transformations, including, for example, oxidation, reduction, hydrogenation, hydrosilylation, hydrocyanation, hydroformylation, polymerization, carbonylation, isomerization, metathesis, carbon-hydrogen activation, carbon-halogen activation, cross-coupling, Friedel-Crafts acylation and alkylation, hydration, Diels-Alder reactions, Baeyer-Villiger reactions, and other transformations. Some compositions, complexes and/or catalysts according to the invention are particularly effective at polymerizing ethylene or α-olefins (such as propylene, 1-butene, 1-pentene, 1-hexene, 1-heptene, 1-octene, 1-decene and styrene), copolymerizing ethylene with α-olefins (such as propylene, 1-butene, 1-pentene, 1-hexene, 1-heptene, 1-octene, and styrene), copolymerizing ethylene with 1,1-disubstituted olefins (such as isobutylene), or copolymerizing ethylene, propylene and a diene monomer suitable for production of EPDM (Ethylene-Propylene-Diene Monomer) synthetic rubbers. Thus, for example, in some embodiments, metal-ligand compositions and complexes containing zirconium or hafnium may be useful in the polymerization of propylene to form isotactic polypropylene or in the copolymerization of ethylene and one or more α-olefins, as noted above. In other embodiments, vanadium and chromium compositions and/or complexes according to the invention may be useful in, for example, the polymerization of ethylene. The compositions, complexes and/or catalysts according to the invention may also polymerize monomers that have polar functionalities in homopolymerizations or copolymerizations and/or homopolymerize 1,1- and 1,2-disubstituted olefins. Also, diolefins in combination with ethylene and/or α-olefins or 1,1- and 1,2-disubstituted olefins may be copolymerized. In some embodiments, catalysts incorporating the ligands, compositions and/or complexes of the present invention exhibit high catalytic activity in the polymerization of such α-olefins, including at high temperatures.

In general, monomers useful herein may be olefinically unsaturated monomers having from 2 to 20 carbon atoms either alone or in combination. Generally, monomers may include olefins (including cyclic olefins), diolefins and unsaturated monomers including ethylene and C₃ to C₂₀ α-olefins such as propylene, 1-butene, 1-hexene, 1-octene, 4-methyl-1-pentene, 1-norbornene, styrene and mixtures thereof; additionally, 1,1-disubstituted olefins, such as isobutylene, 2-methyl-1-butene, 2-methyl-1-pentene, 2-ethyl-1-pentene, 2-methyl-1-hexene, 3-trimethylsilyl-2-methyl-1-propene, α-methyl-styrene, either alone or with other monomers such as ethylene or C₃ to C₂₀ α-olefins and/or diolefins; additionally 1,2-substituted olefins, such as 2-butene. The α-olefins listed above may be polymerized in a stereospecific manner, for example, as in the generation of isotactic or syndiotactic or hemiisotactic polypropylene. Additionally the α-olefins may be polymerized to produce a polymer with differing tacticity sequences within the polymer chain, such as polypropylene containing atactic and isotactic sequences within the same polymer chain. Diolefins generally comprise 1,3-dienes (such as butadiene), substituted 1,3-dienes (such as isoprene), and other substituted 1,3-dienes, with the term substituted referring to the same types of substituents referred to above in the definition section. Diolefins also comprise 1,5-dienes and other non-conjugated dienes, such as ethylidene-norbornene, 1,4-hexadiene, dicyclopentadiene and other dienes used in the manufacture of EPDM synthetic rubbers. The styrene monomers may be unsubstituted or substituted at one or more positions on the aryl ring. The use of diolefins in this invention is typically in conjunction with another monomer that is not a diolefin. In some embodiments, acetylenically unsaturated monomers may be employed.

More specifically, in various embodiments the catalysts of the present invention are active for certain monomers, such as for example ethylene and/or styrene. In particular, the catalysts of the present invention may be used to co-polymerize ethylene and styrene (or substituted styrenes), forming ethylene-styrene copolymers. Exemplary copolymers of ethylene with at least one styrene monomer may comprise from greater than about 0.1 mol. % styrene to less than about 100 mol. % styrene, or from greater than about 0.2 mol. % styrene to less than about 50 mol. % styrene. The catalysts of the present invention are also useful to polymerize, for example, a vinylidene aromatic monomer in a solution polymerization process conducted at a temperature greater than or equal to 100° C., about 120° C. or more (as detailed elsewhere herein).

Polymers that can be prepared according to the present invention include ethylene copolymers with at least one C₃-C₂₀ α-olefin, particularly propylene, 1-butene, 1-hexene, 4-methyl-1-pentene and 1-octene. The copolymers of ethylene with at least one C₃-C₂₀ α-olefin comprise from about 0.1 mol. % α-olefin to about 50 mol. % α-olefin, more specifically from about 0.2 mol. % α-olefin to about 50 mol. % α-olefin and still more specifically from about 2 mol. % α-olefin to about 30 mol. % higher olefin. For certain embodiments of this invention, product copolymers may include those of ethylene and a comonomer selected from the group consisting of propylene, 1-butene, 1-hexene, and 1-octene comprising from about 0.2 to about 30 mol. % comonomer, more specifically from about 1 to about 20 mol. % comonomer. In certain embodiments, ethylene copolymers with at least one C₃-C₂₀ α-olefin can be produced with a low molecular weight (Mw), such as for example molecular weights of less than about 100,000 or, more specifically, less than about 50,000.

The catalysts of the present invention may also be used to prepare a variety of styrene polymers (e.g., homopolymers of styrene and/or substituted styrene). These polymers may be crystalline or amorphous (i.e., polymers that do or do not have a Tm), and may have varying degrees of isotacticity (i.e., varying % MM, ranging for example from about 5% to about 95%, or from about 10% to about 90%, or about 25% to about 85%). These polymers may also have a molecular weight (Mw), for example, of greater than about 200,000, about 400,000 or more.

The ligands, compositions, complexes, and/or catalysts of the invention may also be used to catalyze other (i.e., non-polymerization) transformations. Examples of asymmetric or enantioselective reactions catalyzed by chiral Group 4 catalysts include olefin hydrogenation, olefin epoxidation, olefin isomerization, olefin-pyridine coupling, imine hydrogenation, aldol reactions, imino aldol reactions, epoxidation of allylic alcohols, alkylation of aldehydes, alkylation of imines, Diels-Alder reactions, Baeyer-Villiger reactions, hydroamination/cyclization of amino-alkenes, pinacol coupling of aldehydes, and hydrosilation of imines, ketones, and olefins. In some embodiments, the complexes and catalysts of the invention may be chiral. For example, in some instances, substantially diastereomerically pure or substantially enantiomerically pure complexes may be useful for stereoselective, asymmetric, enantioselective, or diastereoselective reactions or transformations. Thus, in some embodiments, substantially enantiomerically- or diastereomerically-pure complexes, metal-ligand compositions, and catalysts according to the invention may be used as asymmetric catalysts for a range of reactions, including polymerization reactions and other (non-polymerization) reactions, including many reactions useful in organic synthesis. In some embodiments, catalysts incorporating the compositions and complexes of the invention may be used to catalyze the asymmetric production of reaction products with enantiomeric excess (ee) or diastereomeric excess (de) of greater than 90% or greater than 99%. The asymmetric synthesis of chiral organic molecules is an important field, and is critical in the synthesis of many pharmaceuticals and other products. Single enantiomers of a chiral product can be prepared by a variety of techniques, including the resolution of racemates, or the use of substantially enantiomerically pure starting materials from the chiral pool of natural products, but for large scale synthesis the use of enantioselective catalysis is often the most attractive, and most economical, choice. See, e.g., Blaser et al., “Enantioselective Synthesis”, pp. 1131-1149, in Applied Homogeneous Catalysis with Organometallic Compounds, Vol. 3, Cornils, B., & Herrmann, W. (eds.), 2nd Edition, Wiley-VCH, Weinheim, Germany, 2002, and Catalytic Asymmetric Synthesis, Ojima (ed.), VCH Publishers, Inc., New York, 1993, and the references cited therein.

In some embodiments, novel products, such as polymers, copolymers or interpolymers, may be formed having unique physical and/or melt flow properties. Such novel polymers can be employed alone or with other polymers in a blend to form products that may be molded, cast, extruded or spun. End uses for the polymers made with the catalysts of this invention include films for packaging, trash bags, bottles, containers, foams, coatings, insulating devices and household items. Also, such functionalized polymers are useful as solid supports for organometallic or chemical synthesis processes.

Polymerization is carried out under polymerization conditions, including temperatures of from about −100° C. to about 300° C., but in one or more particular embodiments is carried out at a high temperature (e.g., greater than about 120° C., as detailed elsewhere herein). The pressure of the polymerization reaction may range from atmospheric to about 3000 atmospheres. Suspension, solution, slurry, gas phase or high-pressure polymerization processes may be employed with the catalysts and compounds of this invention. Such processes can be run in a batch, semi-batch or continuous mode. Examples of such processes are well known in the art. A support for the catalyst may be employed, which may be inorganic (such as alumina, magnesium chloride or silica) or organic (such as a polymer or cross-linked polymer). Methods for the preparation of supported catalysts are known in the art. Slurry, suspension, gas phase and high-pressure processes as known to those skilled in the art may also be used with supported catalysts of the invention.

As discussed herein, catalytic performance can be determined a number of different ways, as those of skill in the art will appreciate. Catalytic performance can be determined by the yield of polymer obtained per mole of metal complex, which in some contexts may be considered to be activity. The examples provide data for these comparisons.

A solution process may be specified for certain benefits, with the solution process being run at a temperature for example above about 90° C., about 100° C., about 110° C., about 120° C., about 130° C. or more. Suitable solvents for polymerization are non-coordinating, inert liquids. Examples include straight and branched-chain hydrocarbons such as isobutane, butane, pentane, isopentane, hexane, isohexane, heptane, octane, Isopar-E® and mixtures thereof; cyclic and alicyclic hydrocarbons such as cyclohexane, cycloheptane, methylcyclohexane, methylcycloheptane, and mixtures thereof, perhalogenated hydrocarbons such as perfluorinated C₄₋₁₀ alkanes, chlorobenzene, and aromatic and alkyl substituted aromatic compounds such as benzene, toluene, mesitylene, and xylene. Suitable solvents also include liquid olefins which may act as monomers or comonomers including ethylene, propylene, 1-butene, butadiene, cyclopentene, 1-hexene, 1-pentene, 3-methyl-1-pentene, 4-methyl-1-pentene, 1,4-hexadiene, 1-octene, 1-decene, isobutylene, styrene, divinylbenzene, allylbenzene, and vinyltoluene (including all isomers alone or in admixture). Mixtures of the foregoing are also suitable.

Other additives that are useful in a polymerization reaction may be employed, such as scavengers, promoters, modifiers and/or chain transfer agents, such as hydrogen, aluminum alkyls and/or silanes.

The ligands, metal-ligand complexes and compositions of this invention can be prepared and tested for catalytic activity in one or more of the above reactions in a combinatorial fashion. Combinatorial chemistry generally involves the parallel or rapid serial synthesis and/or screening or characterization of compounds and compositions of matter. U.S. Pat. Nos. 5,985,356, 6,030,917 and WO 98/03521, all of which are incorporated herein by reference, generally disclose combinatorial methods. In this regard, the ligands, metal-ligand complexes or compositions may be prepared and/or tested in rapid serial and/or parallel fashion, e.g., in an array format. When prepared in an array format, ligands, metal-ligand complexes or compositions may take the form of an array comprising a plurality of compounds wherein each compound can be characterized by any of the above general formulas (I.e., I, II, etc.). An array of ligands may be synthesized using the procedures outlined previously. The array may also be of metal precursor compounds, the metal-ligand complexes or compositions characterized by the previously described formulae and/or description. Typically, each member of the array will have differences so that, for example, a ligand or activator or metal precursor or R group in a first region of the array may be different than the ligand or activator or metal precursor or R group in a second region of the array. Other variables may also differ from region to region in the array.

In such a combinatorial array, typically each of the plurality of compositions or complexes has a different composition or stoichiometry, and typically each composition or complex is at a selected region on a substrate such that each compound is isolated from the other compositions or complexes. This isolation can take many forms, typically depending on the substrate used. If a flat substrate is used, there may simply be sufficient space between regions so that there cannot be interdiffusion between compositions or complexes. As another example, the substrate can be a microtiter or similar plate having wells so that each composition or complex is in a region separated from other compounds in other regions by a physical barrier. The array may also comprise a parallel reactor or testing chamber.

The array typically comprises at least 8 compounds, complexes or compositions each having a different chemical formula, meaning that there must be at least one different atom or bond differentiating the members in the array or different ratios of the components referred to herein (with components referring to ligands, metal precursors, activators, group 13 reagents, solvents, monomers, supports, etc.). In other embodiments, there are at least 20 compounds, complexes or compositions on or in the substrate each having a different chemical formula. In still other embodiments, there are at least 40 or 90 or 124 compounds, complexes or compositions on or in the substrate each having a different chemical formula. Because of the manner of forming combinatorial arrays, it may be that each compound, complex or composition may not be worked-up, purified or isolated, and for example, may contain reaction by-products or impurities or unreacted starting materials.

The catalytic performance of the compounds, complexes or compositions of this invention can be tested in a combinatorial or high throughput fashion. Polymerizations can also be performed in a combinatorial fashion, see, e.g., U.S. Pat. Nos. 6,306,658, 6,508,984 and WO 01/98371, each of which is herein incorporated by reference.

### EXAMPLES

All air sensitive reactions were performed under a purified argon or nitrogen atmosphere in a Vacuum Atmospheres or MBraun glove box. All solvents used were anhydrous, de-oxygenated and purified according to known techniques. All ligands and metal precursors were prepared according to procedures known to those of skill in the art, e.g., under inert atmospheric conditions, etc. Unless otherwise indicated, polymerizations were generally carried out in a parallel pressure reactor, which is described in U.S. Pat. Nos. 6,306,658, 6,455,316 and 6,489,168, and WO 00/09255, each of which is incorporated herein by reference. The above-described analytical techniques were utilized, generally.

### Example 1

### Ligand Synthesis

### Section 1A. Synthesis of 3-Phenol 1,2,4-Oxadiazoles, C-C Coupled Ligands

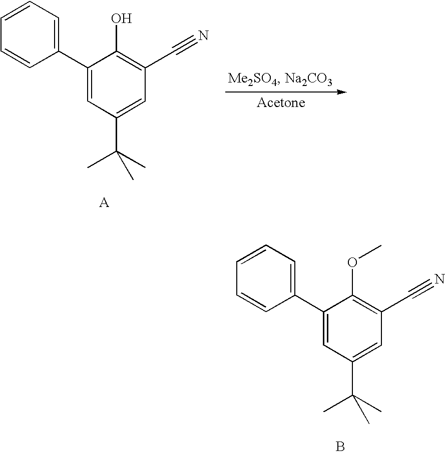

To begin, Me₂SO₄ (336 mg, 2.67 mmoles, 252 μL) and K₂CO₃ (566 mg, 4.10 mmoles) were added to a solution of phenol nitrile (A) (515 mg, 2.05 mmoles) in 8 mL of acetone at room temperature. The suspension was heated to 60° C. for 4 hours. After cooling, the reaction was quenched with saline and extracted 3 times with diethyl ether. The aqueous phase was then discarded. The material was dried over Na₂SO₄, filtered, and concentrated to dryness. The crude product (567 mg) was used directly in the next reaction.

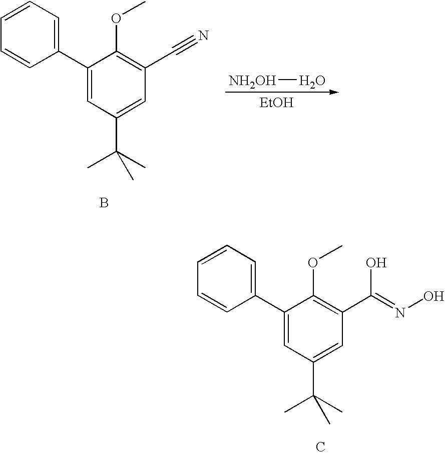

In the second reaction, NH₂OH—H₂O (50% wt. In H₂O, 541 mg, 8.20 mmoles) was added to a solution of methyl protected phenol nitrile B (567 mg) in 5 mL of EtOH at room temperature The mixture was heated to 80° C. with stirring for 3 hours. After cooling, the reaction was quenched with saline and extracted 3 times with EtOAc. The aqueous phase was then discarded. The material was dried over Na₂SO₄, filtered, and concentrated to dryness. The crude product C (574 mg) was used directly in a third reaction.

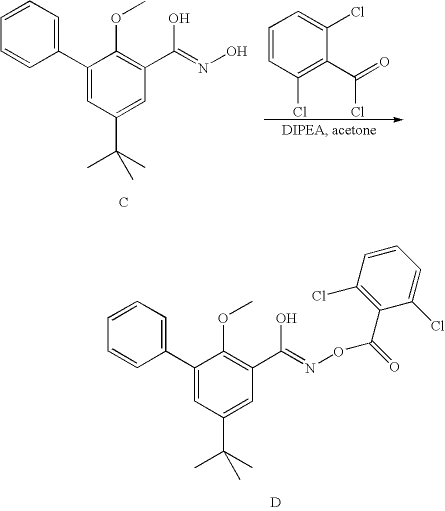

The crude product C (278 mg) was next dissolved in acetone (8 mL) followed by the addition of 2,6-diCl-PhCOCl (195 mg; 923 μmoles) and Diisopropylethylamine (120 mg; 933 μmoles; 162 μL) at room temperature. The mixture was held at room temperature for 3 hours with stirring. The reaction was quenched by saturating the mixture in NaHCO₃/H₂O. The mixture was then extracted 3 times with diethyl ether and the aqueous phase was discarded. The material was dried over Na₂SO₄, filtered, and concentrated to dryness. The crude mixture was purified by flash chromatography on silica. The column was eluted with 10-20% (by weight) EtOAc/Hex. The appropriate fractions were then combined and concentrated to form product D in 70% yield over 3 steps (329 mg; 698 μmoles).

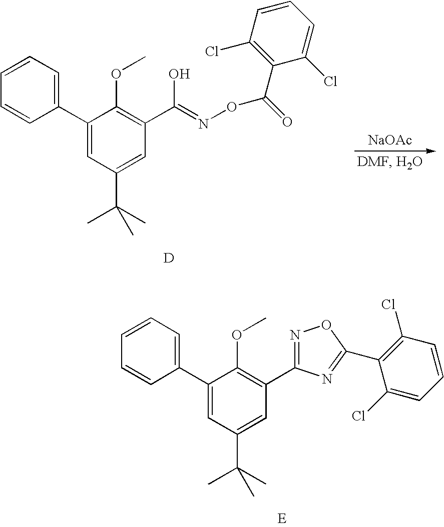

In another step, NaOAc (14 mg; 170 μmoles) was added to a solution of acyl amide oxime D (67 mg, 142 μmoles) in DMF (2 mL) and water (100 μL) at room temperature. The mixture was heated to 100° C. with stirring for 16 hours. The reaction was quenched with saline. The mixture was extracted 3 times with diethyl ether and the aqueous phase was discarded. The material was then dried over Na₂SO₄, filtered, and concentrated to dryness. The crude mixture was purified by flash chromatography on silica. The column was eluted with 3-10% (by weight) EtOAc/Hex; the appropriate fractions were then combined and concentrated to produce E in 41% yield (26 mg, 57 μmoles).

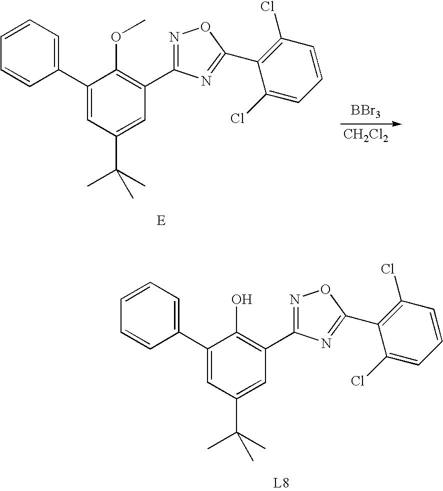

Methyl protected phenol E (39 mg, 86 μmoles) was then dissolved in DCM (5 mL). Boron Tribromide (1.0 M in CH₂Cl₂, 258 μL; 258 μmoles) was added to the vessel containing the solution of E with stirring and the resulting mixture was kept at room temperature for 3 hours. The reaction was quenched by saturating the mixture with NaHCO₃/H₂O. The mixture was then extracted 3 times with diethyl ether and the aqueous phase was discarded. The material was dried over Na₂SO₄, filtered, and concentrated to dryness. The crude mixture was purified by flash chromatography on silica. The column was eluted with 3-10% (by weight) EtOAc/Hex. The appropriate fractions were then combined and concentrated to form product L8 in 58% yield (22 mg; 50 μmoles).

Section 1B. Synthesis of 3-Phenol 1,2,4-Oxadiazoles, C-N Coupled Ligands

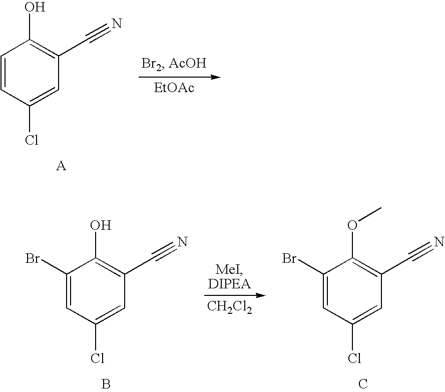

A solution of 2-CN-4-Cl Phenol (A) (5 mmoles; 1.00 equiv; 767.84 mg), Acetic Acid (10 mmoles; 10.00 mmoles; 573.02 μL) and Ethyl Acetate (20 mL; 204.39 mmoles; 20.00 mL) was charged in a 40 mL Screw-cap Vial. Bromine (10 mmoles; 513.85 μL) was then added drop-wise to the reaction at room temperature. The mixture was heated to 60° C. with stirring and held for 12 hours. The reaction was quenched with aqueous Na₂S₂O₃. The mixture was then extracted 3 times with ethyl acetate and the aqueous phase was discarded. The product (B) was dried over Na₂SO₄, filtered, and concentrated to dryness. The residue was dissolved in 10 mL CH₂Cl₂. Diisopropylethylamine (5.5 mmoles; 959.18 μL) and methyl iodide (5.5 mmoles; 342.55 μL) were added to the residue solution. The mixture was heated to 40° C. with stirring and held for 12 hours. The reaction was quenched with aqueous NH₄Cl. The mixture was then extracted 3 times with dichloromethane and the aqueous phase was discarded. The material was then dried over Na₂SO₄, filtered, and concentrated to dryness. The crude mixture was purified by flash chromatography on silica. The column was eluted with 1-15% (by weight) EtOAc/Hex to give C in 81% yield (1 g; 4.06 mmoles).

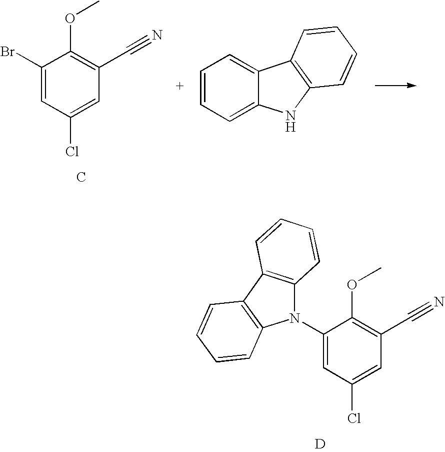

The second reaction was operated in dry box. Specifically, Bromo methyl protected phenol C (3 mmoles; 739.47 mg), Cbz (3 mmoles; 501.63 mg), 1,2-Cyclohexanediamine (69 mg; 604.25 μmoles; 74.18 μL); Tetrakis(acetonitrile)copper(1) Hexafluorophosphate (112 mg; 294.48 μmoles), Potassium Phosphate, Tribasic, N-Hydrate (765 mg; 3.60 mmoles) and p-Xylene (6 mL; 48.67 mmoles) were added to a 20 mL Screw-cap Vial. The mixture was heated to 120° C. with stirring for 16 hours. The material was then filtered and washed with dichloromethane (CH₂Cl₂). The material was concentrated by rotovap. The crude mixture was purified by flash chromatography on silica. The column was eluted with 10-25% (by weight) EtOAc/Hex. The appropriate fractions were then combined and concentrated to give D in 24% yield (233 mg; 700.14 μmoles).

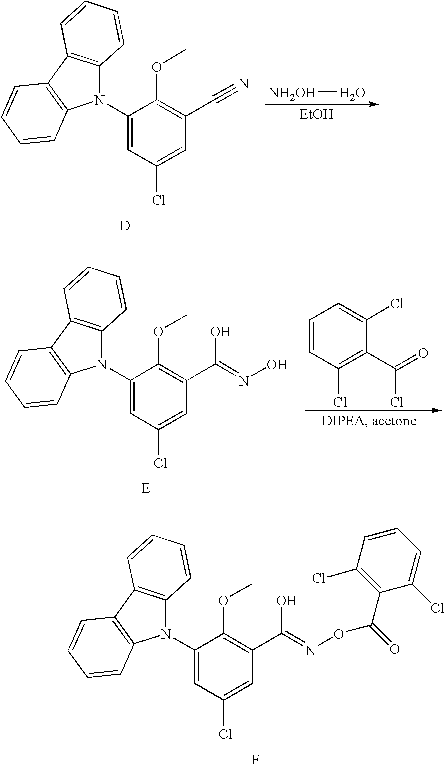

In a subsequent reaction, a single portion of Cbz-methyl protected phenol nitrile D (233 mg; 700.14 μmoles), NH₂OH.H₂O (0.25 mL; 3.78 mmoles) and Ethanol (6 mL; 103.06 mmoles) was added to a 20 mL Screw-cap Vial. The mixture was heated to 80° C. with stirring for 3 hours. After the mixture was cooled, the reaction was quenched with water. The mixture was extracted 3 times with dichloromethane (DCM) and the aqueous phase was discarded. The crude product E was dried over Na₂SO₄, filtered, and concentrated to dryness. The crude product E was dissolved in acetone (6 mL; 81.62 mmoles), followed by the addition of 2,6-diCl-PhCOCl (146 mg; 697.04 μmoles) and Diisopropylethylamine (89 mg; 688.61 μmoles; 120.09 μL). The mixture was held with stirring at room temperature for 3 hours. The reaction was quenched with water. The mixture was extracted 3 times with diethyl ether and the aqueous phase was discarded. The material was dried over Na₂SO₄, filtered, and concentrated to dryness. The crude mixture was then purified by flash chromatography on silica. The column was eluted with 5-20% (by weight) EtOAc/Hex. The appropriate fractions were combined and concentrated to form product E in 85% yield (311 mg; 597.17 μmoles).

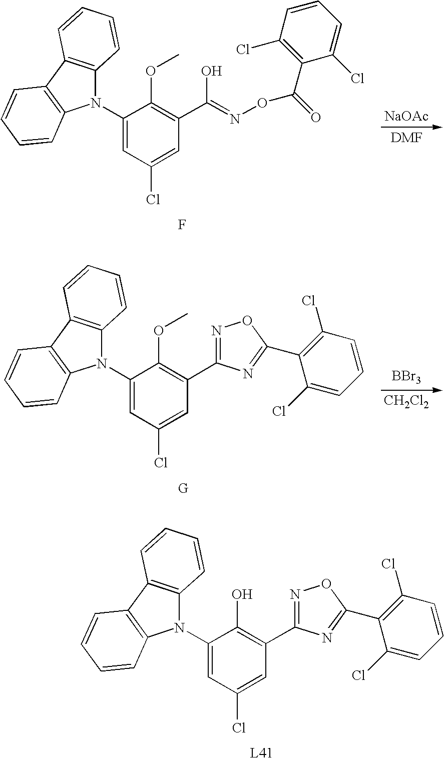

Subsequently, compound F (311 mg; 597.17 μmoles; 311.00 mg) and sodium ethanoate (68 mg; 828.92 μmoles) were added to a solution of dimethylformamide (5 mL; 64.66 mmoles; 5.00 mL) and water (0.2 mL) at room temperature. The mixture was heated to 100° C. with stirring for 16 hours. The reaction was quenched with water. The mixture was extracted 3 times with diethyl ether and the aqueous phase was discarded. The material was dried over Na₂SO₄, filtered, and concentrated to dryness. The crude mixture was purified by flash chromatography on silica. The column was then eluted with 1-10% (by weight) EtOAc/Hex. The appropriate fractions were then combined and concentrated to give G.

The residue was dissolved in DCM (5 mL). Boron Tribromide (0.8 mL; 800.00 μmoles) was then added to the vessel with stirring and the resulting mixture was kept at room temperature for 3 hours. The reaction was quenched with methanol. The material was then dried in a vacuum, passed through a silica gel plunger, to give a white solid as final product L41 in 55% yield (2 steps) (168 mg; 331.51 μmoles).

### Example 2

### Complexation

### Section 2A. Synthesis of M1

To begin, 2 mL of an orange toluene solution of ZrBz₄ (23.4 mM) was added dropwise over a period of 5 minutes to a 4 mL colourless toluene solution of L8 (23.4 mM). The solution was allowed to stand for two hours at room temperature. The solution was then filtered, blown down to approximately 0.5 mL, and then transferred into a 4-mL vial within a 20-mL vial, half-way filled with pentane. The vial was placed in a freezer at −35° C. overnight. The following morning, large crystals had formed and were isolated. The final product was an orange crystalline solid, yield 60% (32 mg). The x-ray crystal structure of the compound is illustrated in FIG. 1 herein.

### Section 2B. Synthesis of M3

To produce M3, 2 mL of an orange toluene solution of ZrBz₄ (10.0 mM) was added dropwise over a period of 2 minutes to a 4 mL colourless toluene solution of L12 (10.0 mM). The resulting dark orange solution was filtered and the solvent was subsequently removed via inert gas. The orange solid was dissolved in approximately 1 mL of benzene and placed in the freezer at −35° C. for 16 hours. The resulting frozen solution was then placed under vacuum for 30 minutes and the solvent was removed. The finial product was an orange solid, yield 85% (23 mg).

### Section 2C. Synthesis of M6

In this synthesis, 1 mL of a yellow toluene solution of HfBz₄ (15.4 mM) was added dropwise over a period of 2 minutes to a 0.5 mL colourless toluene solution of L6 (61.7 mM). The resulting dark orange solution was filtered and the solvent was subsequently removed via inert gas. The orange solid was dissolved in approximately 1 mL of benzene and placed in the freezer at −35° C. for 16 hours. The resulting frozen solution was placed under vacuum for 30 minutes and the solvent was removed. The finial product was a yellow solid, yield 97% (20 mg).

### Example 3

### Polymerization

Section 3A. Styrene Polymerization with in-situ Generated Complexes

Ligand arrays (0.5-2.0 μmol of each ligand source, 2 eq. vs. Zr) were charged with toluene (100 μL per well) and then toluene solutions of zirconium tetrabenzyl metal precursor (15-60 μL per well, 0.25-1.0 μmol) were added. The resultant mixtures were stirred for 45 minutes at 75° C. within an aluminum block (pre-mix array). The pre-mix array was allowed to cool to 25° C. with stirring. Individual vials were then treated with a stock solution of PMAO-IP (scavenger) (30-60 μL per well, 5:1 equivalent ratio based on metal complex, contact time 1 min, 25° C.), followed by [HN(C₁₀H₂₁)₂(p-Bu-Ph)]+[B(C₆F₅)₄]⁻ (30-75 μL per well, 1:1 equivalent ratio based on metal complex, contact time 1 min, 25° C.). Individual aliquots of the resulting solutions were transferred to 15 mL tarred glass vials containing: polytetrafluoroethylene coated stir bars, 2 mL of ethylbenzene, PMAO-IP (10 μmol) and 2 mL of styrene (added 5 minutes before addition of the above aliquots in order to minimize auto-polymerization generated PS). The vials were mounted in a temperature controlled 4×3 reactor array and heated to 125° C. After 5 minutes following addition of the catalyst solutions, the vials were removed from the reactor array, placed in an Al array (at ambient temperature), and 2 ml of a 10:1 (v:v) ethylbenzene:1-nonanol were added to each vial. The Al array holding the mL vials was then removed from the glove box; transferred to a fume hood; and each vial received 5 mL of methanol. Volatiles were removed from the vials by use of a Genevac™ (for 16 hours). The vials were then further dried within a heated vacuum oven followed by weighting for a minimum of 2*2-4 hour cycles until constant weights were attained.

Table 3A presents the results form the styrene polymerization reactions performed.

TABLE 3A
Results of Styrene Polymerization Experiments
with in-situ Generated Complexes

| | | | | | | | | tacticity |
|---|---|---|---|---|---|---|---|---|
| | | | | | | | | index |
| | | in-situ | | | | | | (% mm |
| run | | complex | yield | | | | | via |
| number | ligand | (μmol) | (g) | conversion | activity* | Mw | PDI | Raman) |
| PS1 | L2 | 0.6675 | 0.2105 | 12 | 63 | 117688 | 2.1 | 86 |
| PS2 | L7 | 0.125 | 0.3138 | 17 | 502 | 207053 | 1.6 | 85 |
| PS3 | L8 | 0.125 | 0.1875 | 10 | 300 | 184479 | 1.6 | 78 |
| PS4 | L9 | 0.2 | 0.2627 | 14 | 263 | 234006 | 2.5 | 73 |
| PS5 | L10 | 0.2 | 0.1891 | 10 | 189 | 213237 | 2.8 | 67 |
| PS6 | L12 | 0.2 | 0.6155 | 34 | 616 | 474140 | 2.5 | 88 |
| PS7 | L14 | 0.2 | 0.5211 | 29 | 521 | 272364 | 2.3 | 84 |
| PS8 | L15 | 0.2 | 0.739 | 41 | 739 | 355969 | 2.4 | 93 |
| PS9 | L18 | 0.2 | 0.0471 | 3 | 47 | 118382 | 13.4 | 28 |
| PS10 | L20 | 0.2 | 0.3698 | 20 | 370 | 145041 | 2.3 | 81 |
| PS11 | L21 | 0.2 | 0.7663 | 42 | 766 | 232639 | 1.9 | 91 |
| PS12 | L22 | 0.2 | 0.3687 | 20 | 369 | 196702 | 1.9 | 82 |
| PS13 | L23 | 0.25 | 0.2899 | 16 | 232 | 133082 | 1.9 | 65 |
| PS14 | L24 | 0.4 | 0.1925 | 11 | 96 | 208000 | 2.0 | 61 |
| PS15 | L25 | 0.1 | 0.3154 | 17 | 631 | 250180 | 1.7 | 90 |
| PS16 | L26 | 0.1 | 0.3555 | 20 | 711 | 190710 | 1.6 | 89 |
| PS17 | L27 | 0.5 | 0.5074 | 28 | 203 | 148422 | 1.5 | 78 |
| PS18 | L28 | 0.5 | 0.6646 | 37 | 266 | 125738 | 1.5 | 83 |
| PS19 | L30 | 0.25 | 0.771 | 43 | 617 | 128926 | 1.5 | 85 |
| PS20 | L31 | 0.25 | 0.4972 | 27 | 398 | 157554 | 1.6 | 78 |
| PS21 | L33 | 0.45 | 0.656 | 36 | 292 | 161838 | 1.5 | 88 |
| PS22 | L34 | 0.45 | 0.3408 | 19 | 151 | 112655 | 1.5 | 86 |
| PS23 | L39 | 0.25 | 1.2148 | 67 | 972 | 252884 | 1.6 | 94 |
| PS24 | L40 | 0.25 | 0.3669 | 20 | 294 | 450630 | 1.5 | 98 |
| PS25 | L59 | 0.25 | 0.4037 | 22 | 323 | 189675 | 1.9 | 81 |
| PS26 | L60 | 0.1 | 0.315 | 17 | 630 | 176530 | 1.6 | 84 |

*(mg of poly./μmol of cat. * min)

Section 3B. Styrene Polymerization with Isolated Generated Complexes

Isolated complexes were pre-mixed (i.e. “pre-mix” runs): (i) with 5 eq. Al(ⁱBu)₃ for 5 minutes, followed by the addition of 1 eq. of NCA=[HN(C₁₀H₂₁)₂(p-Bu-Ph)]⁺[B(C₆F₅)₄]⁻; or (ii) with 500 eq. MMAO-3A for 1 minute; individual aliquots of the resulting solutions were injected into 15 ml vials within a temperature controlled 4×3 reactor array. Alternatively, in some of the runs (i.e., “in-reactor” runs), the 500 eq. MMAO-3A was added the same reactor followed immediately by the addition of the isolated complex solution. The temperature controlled 4×3 reactor array housed 15 mL tarred glass vials containing polytetrafluoroethylene coated stir bars. Three run conditions were examined: (i) 125° C., 2 mL of ethylbenzene, PMAO-IP (10 μmol) and 2 mL of styrene (17396 μmol); (ii) 125° C., PMAO-IP (10 μmol) and 4 mL of styrene (34792 μmol); (iii) 145° C., 2 mL of t-butylbenzene, PMAO-IP (10 μmol) and 2 mL of styrene (17396 μmol); in all instants, the styrene was added 5 minutes before addition of the above aliquots in order to minimize auto-polymerization generated PS. Post-experiment duration (5-15 minutes), reaction solutions were handled as described in Section 3A.

Table 3B presents the results from the styrene polymerization reactions performed with isolated complexes.

TABLE 3B
Results of Styrene Polymerization Experiments with Isolated Complexes

| | | | | | | | | | | | | | | | tacticity |
|---|---|---|---|---|---|---|---|---|---|---|---|---|---|---|---|
| | | isolated | | | | | reaction | | | | | | | | index |
| | isolated | com- | Al | | | | tem- | expt. | | | con- | | | | (% mm |
| | com- | plex | re- | | | activation | perature | time | styrene | yield | ver- | | | | via |
| run | plex | (μmol) | agent | activator | scavenger^{a} | mode^{b} | (° C.) | (min) | (μmol) | (g) | sion | activity^{c} | Mw | PDI | Raman) |
| PS36 | M3 | 0.075 | | MMAO-3A | PMAO-IP | in-reactor | 125 | 5 | 17396 | 0.3457 | 19 | 922 | 376610 | 1.7 | 89 |
| PS37 | M3 | 0.075 | | MMAO-3A | PMAO-IP | in-reactor | 125 | 5 | 34792 | 0.6917 | 19 | 1845 | 378393 | 1.8 | 84 |
| PS38 | M3 | 0.075 | | MMAO-3A | PMAO-IP | in-reactor | 145 | 5 | 17396 | 0.5684 | 31 | 1516 | 192064 | 1.6 | 85 |
| PS39 | M3 | 0.075 | | MMAO-3A | PMAO-IP | pre-mix | 145 | 5 | 17396 | 0.4393 | 24 | 1171 | 201257 | 1.7 | 71 |
| PS40 | M3 | 0.15 | TIBA | NCA | PMAO-IP | pre-mix | 125 | 5 | 17396 | 0.4932 | 27 | 658 | 355563 | 1.9 | |
| PS41 | M3 | 0.036 | TIBA | NCA | PMAO-IP | pre-mix | 125 | 5 | 34792 | 0.1589 | 4 | 883 | 392318 | 1.7 | |
| PS42 | M3 | 0.075 | TIBA | NCA | PMAO-IP | pre-mix | 145 | 5 | 17396 | 0.3396 | 19 | 906 | | | 75 |
| PS43 | M4 | 0.075 | | MMAO-3A | PMAO-IP | in-reactor | 125 | 5 | 17396 | 0.319 | 18 | 851 | 327959 | 1.7 | 89 |
| PS44 | M4 | 0.075 | | MMAO-3A | PMAO-IP | in-reactor | 125 | 5 | 34792 | 0.5357 | 15 | 1429 | 345307 | 1.8 | 79 |
| PS45 | M4 | 0.15 | TIBA | NCA | TIBA | pre-mix | 125 | 5 | 17396 | 0.2965 | 16 | 395 | 333182 | 1.8 | 89 |
| PS46 | M4 | 0.036 | TIBA | NCA | PMAO-IP | pre-mix | 125 | 5 | 34792 | 0.1254 | 3 | 697 | 379149 | 1.7 | |
| PS47 | M5 | 0.075 | | MMAO-3A | PMAO-IP | in-reactor | 125 | 5 | 17396 | 0.3188 | 18 | 850 | 278285 | 1.7 | 90 |
| PS48 | M5 | 0.075 | | MMAO-3A | PMAO-IP | in-reactor | 125 | 5 | 34792 | 0.5743 | 16 | 1531 | 311690 | 1.8 | 79 |
| PS49 | M5 | 0.15 | TIBA | NCA | TIBA | pre-mix | 125 | 5 | 17396 | 0.3438 | 19 | 458 | 283231 | 1.7 | 91 |
| PS50 | M5 | 0.036 | TIBA | NCA | PMAO-IP | pre-mix | 125 | 5 | 34792 | 0.148 | 4 | 822 | 359606 | 1.7 | |
| PS51 | M8 | 0.036 | | MMAO-3A | PMAO-IP | in-reactor | 125 | 5 | 17396 | 0.2467 | 14 | 1371 | 522527 | 1.7 | |
| PS52 | M8 | 0.036 | | MMAO-3A | PMAO-IP | in-reactor | 125 | 5 | 34792 | 0.5217 | 14 | 2898 | 514284 | 2.0 | |
| PS53 | M8 | 0.036 | | MMAO-3A | PMAO-IP | in-reactor | 145 | 5 | 17396 | 0.3848 | 21 | 2138 | 245245 | 1.7 | 80 |
| PS54 | M8 | 0.036 | | MMAO-3A | PMAO-IP | pre-mix | 145 | 5 | 17396 | 0.2392 | 13 | 1329 | 231510 | 1.7 | |
| PS55 | M8 | 0.15 | TIBA | NCA | PMAO-IP | pre-mix | 125 | 5 | 17396 | 0.784 | 43 | 1045 | 398785 | 1.7 | |
| PS56 | M8 | 0.075 | TIBA | NCA | PMAO-IP | pre-mix | 145 | 5 | 17396 | 0.5321 | 29 | 1419 | | | 72 |
| PS57 | M9 | 0.075 | | MMAO-3A | PMAO-IP | in-reactor | 125 | 5 | 17396 | 0.3017 | 17 | 805 | 344128 | 1.7 | |
| PS58 | M9 | 0.075 | | MMAO-3A | PMAO-IP | in-reactor | 125 | 5 | 34792 | 1.0293 | 28 | 2745 | 364881 | 1.9 | |
| PS59 | M9 | 0.15 | TIBA | NCA | PMAO-IP | pre-mix | 125 | 15 | 17396 | 0.1881 | 10 | 84 | 332461 | 2.2 | |
| PS60 | M10 | 0.3 | | MMAO-3A | PMAO-IP | in-reactor | 125 | 5 | 17396 | 0.1733 | 10 | 116 | 576110 | 1.7 | |
| PS61 | M10 | 0.3 | | MMAO-3A | PMAO-IP | in-reactor | 125 | 5 | 34792 | 0.3861 | 11 | 257 | 698741 | 2.6 | |
| PS62 | M10 | 0.3 | | MMAO-3A | PMAO-IP | in-reactor | 145 | 15 | 17396 | 0.4234 | 23 | 94 | 280604 | 3.6 | 84 |
| PS63 | M10 | 0.3 | TIBA | NCA | PMAO-IP | pre-mix | 125 | 5 | 17396 | 0.1041 | 6 | 69 | 454024 | 2.5 | |
| PS64 | M10 | 0.3 | TIBA | NCA | PMAO-IP | pre-mix | 145 | 15 | 17396 | 0.2287 | 13 | 51 | | | |
| PS65 | M11 | 0.075 | | MMAO-3A | PMAO-IP | in-reactor | 125 | 5 | 17396 | 0.3176 | 18 | 847 | 365193 | 1.9 | 93 |
| PS66 | M11 | 0.075 | | MMAO-3A | PMAO-IP | in-reactor | 125 | 5 | 34792 | 0.5667 | 16 | 1511 | 361492 | 1.8 | |
| PS67 | M11 | 0.15 | TIBA | NCA | PMAO-IP | pre-mix | 125 | 5 | 17396 | 0.2184 | 12 | 291 | 319370 | 1.7 | 93 |
| PS68 | M11 | 0.036 | TIBA | NCA | PMAO-IP | pre-mix | 125 | 5 | 34792 | 0.1356 | 4 | 753 | 351742 | 1.7 | |
| PS69 | M12 | 0.075 | | MMAO-3A | PMAO-IP | in-reactor | 125 | 5 | 17396 | 0.5443 | 30 | 1451 | 206970 | 1.7 | 90 |
| PS70 | M12 | 0.075 | | MMAO-3A | PMAO-IP | in-reactor | 125 | 5 | 34792 | 1.1001 | 30 | 2934 | 230549 | 1.8 | 88 |
| PS71 | M12 | 0.075 | TIBA | NCA | PMAO-IP | pre-mix | 125 | 5 | 17396 | 0.2898 | 16 | 773 | 228185 | 1.7 | 87 |
| PS72 | M12 | 0.036 | TIBA | NCA | PMAO-IP | pre-mix | 125 | 5 | 34792 | 0.2912 | 8 | 1618 | 256175 | 1.7 | 71 |
| PS73 | no | | | | PMAO-IP | | 145 | 5 | 17396 | 0.0531 | 3 | | 176126 | 1.5 | <60 |
| | com- | | | | | | | | | | | | | | |
| | plex | | | | | | | | | | | | | | |

^{a}10 μmol of reactor scavenger
^{b}pre-mix v. ‘in-reactor’ activation
^{c}mg of poly./μmol of cat. * min)

Section 3C. Ethylene-Styrene Co-polymerization with Isolated Complexes

Isolated complexes were first pre-mixed with 5 eq. Al(ⁱBu)₃ for 1 minute, followed by the addition of 1 eq. of NCA=[HN(C₁₀H₂₁)₂(p-Bu-Ph)]⁺[B(C₆F₅)₄]⁻. An aliquot of the resulting solution was injected into a reactor (4.5 ml total volume). The reactor was then run with: 100 psig ethylene, and 10 μmol PMAO-IP, as in-reactor scavenger, at a temperature of 105° C. for polymerization.

Table 3C presents the results from the ethylene-styrene co-polymerization reactions performed with isolated complexes.

TABLE 3C
Results of Ethylene-Styrene Co-polymerization Experiments with
Isolated Complexes

| | | | | | | | | | | wt % |
|---|---|---|---|---|---|---|---|---|---|---|
| | | isolated | expt. | | | | | | | styrene |
| run | isolated | complex | time | styrene | yield | | | | | (1H |
| number | complex | (μmol) | (sec) | (μmol) | (g) | activity* | Mw | Mn | PDI | NMR) |
| ES1 | M1 | 0.075 | 1800 | 4349 | 0.0954 | 42 | 25729 | 4165 | 6.2 | 17.5 |
| ES2 | M1 | 0.06 | 1801 | 8698 | 0.1224 | 68 | 40431 | 2901 | 13.9 | 21.1 |
| ES3 | M8 | 0.075 | 1345 | 4349 | 0.1243 | 74 | 1858 | 1349 | 1.4 | 43.1 |
| ES4 | M8 | 0.1 | 1801 | 8698 | 0.2019 | 67 | 2047 | 1405 | 1.5 | 60.7 |

*(mg of poly./μmol of cat. * min)

Section 3D. Ethylene Homo & Ethylene-Octene Co-Polymerization with Isolated Complexes

Isolated complexes were first contacted with a heptane slurry of MAO on silica at a 19 μmol cat./g support for approximately 2-3 hours. An aliquot of the resulting solution was injected into a reactor (5 ml total volume). The reactor was then run with: 120 psig ethylene, with heptane as a solvent, and 10 μmol triotylaminum, as in-reactor scavenger, at a temperature of 85° C. for polymerization.

Table 3D presents the results from the ethylene homo and ethylene-octene co-polymerization reactions performed with isolated complexes.

TABLE 3D
Results of Ethylene Homo & Ethylene-Octene Co-polymerization
Experiments with Isolated Complexes

| | | isolated | | | | | | |
|---|---|---|---|---|---|---|---|---|
| run | isolated | complex | expt. time | octene | yield | | Mw | |
| number | complex | (μmol) | (sec) | (μl) | (g) | activity* | (k) | PDI |
| EO1 | M6 | 0.15 | 1626 | 0 | 0.0403 | 115 | 70 | 1.8 |
| EO2 | M6 | 0.10 | 1666 | 65 | 0.0382 | 106 | 45 | 1.8 |
| EO3 | M7 | 0.10 | 1408 | 0 | 0.0506 | 290 | 62 | 2.0 |
| EO4 | M7 | 0.07 | 1391 | 65 | 0.0431 | 250 | 50 | 1.9 |

*(mg of poly./μmol of cat. * min)

Although the present invention has been described with reference to preferred embodiments, persons skilled in the art will recognize that changes may be made in form and detail without departing from the spirit and scope of the invention.

When introducing elements of the present disclosure or the embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A compound that is a metal ligand complex characterized by the general formula: 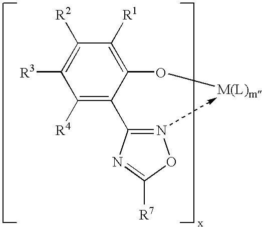
wherein:
each of R¹, R², R³ and R⁴ are the same or different and are independently selected from the group consisting of hydrogen, alkyl, substituted alkyl, cycloalkyl, substituted cycloalkyl, heteroalkyl, substituted heteroalkyl, heterocycloalkyl, substituted heterocycloalkyl, aryl, substituted aryl, heteroaryl, substituted heteroaryl, alkoxy, aryloxy, halo, silyl, boryl, phosphino, amino, thioalkyl, thioaryl, nitro, and combinations thereof, with the proviso that (i) at least one of R¹, R², R³ and R⁴ is selected from the group consisting of carbazolyl and substituted carbazolyl; and further (ii) at least one of R¹, R², R³ and R⁴ is selected from the group consisting of alkyl, substituted alkyl, halo, and alkoxy;
R⁷ is selected from the group consisting of phenyl, substituted phenyl, and anthracenyl;
M is a metal selected from the group consisting of groups 3 through 6 of the periodic table elements and lanthanides;
each L is independently selected from the group consisting of hydrogen, halogen, optionally substituted alkyl, heteroalkyl, allyl, diene, alkenyl, heteroalkenyl, alkynyl, heteroalkynyl, aryl, heteroaryl, alkoxy, aryloxy, boryl, silyl, amino, phosphino, ether, thioether, phosphine, amine, carboxylate, alkylthio, arylthio, 1,3-dionate, oxalate, carbonate, nitrate, sulphate, and combinations thereof;
x is 1, 2, 3, or 4; and
m″ is 0, 1, 2, 3, or 4.

2. The compound of claim 1, wherein the metal ligand complex is characterized by a formula 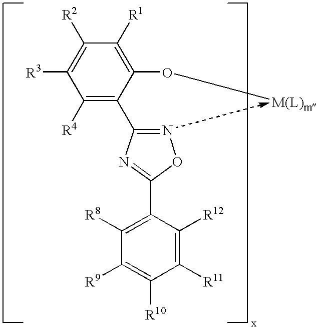
wherein:
R¹, R², R³, and R⁴ are as defined in claim 1;
R⁸, R⁹, R¹⁰, R¹¹, R¹² are the same or different and are independently selected from the group consisting of hydrogen, alkyl, substituted alkyl, cycloalkyl, substituted cycloalkyl, heteroalkyl, substituted heteroalkyl, heterocycloalkyl, substituted heterocycloalkyl, aryl, substituted aryl, heteroaryl, substituted heteroaryl, alkoxy, aryloxy, halo, silyl, boryl, phosphino, amino, thioalkyl, thioaryl, nitro, and combinations thereof, and optionally two or more of R⁸, R⁹, R¹⁰, R¹¹, and R¹² may be joined to form a fused ring system having up to 50 atoms, not counting hydrogen atoms.

3. The compound of claim 1, wherein R¹ is selected from the group consisting of carbazolyl and substituted carbazolyl.

4. The compound of claim 3, wherein R⁷ is selected from the group consisting of substituted phenyl and anthracenyl.

5. The compound of claim 4, wherein R¹ is selected from the group consisting of N-carbazolyl and substituted N-carbazolyl; R³ is selected from the group consisting of halo and tBu, and R⁷ is selected from the group consisting of substituted phenyl and anthracenyl.

6. The compound of claim 5 wherein R¹ is N-carbazolyl; R³ is tBu; and R⁷ is dihalophenyl.

7. The compound of claim 5 wherein R¹ is N-carbazolyl; R³ is halo; and R⁷ is dihalophenyl.

8. The compound of claim 1, wherein x is 2, forming a bis-ligand complex, and further wherein each of the two ligands within the square brackets is identical to the other.

9. A composition comprising:
a) a compound characterized by the general formula: 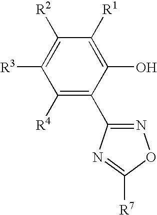
wherein
each of R¹, R², R³ and R⁴ are the same or different and are independently selected from the group consisting of hydrogen, alkyl, substituted alkyl, cycloalkyl, substituted cycloalkyl, heteroalkyl, substituted heteroalkyl, heterocycloalkyl, substituted heterocycloalkyl, aryl, substituted aryl, heteroaryl, substituted heteroaryl, alkoxy, aryloxy, halo, silyl, boryl, phosphino, amino, thioalkyl, thioaryl, nitro, and combinations thereof, with the proviso that (i) at least one of R¹, R², R³ and R⁴ is selected from the group consisting of carbazolyl and substituted carbazolyl; and further (ii) at least one of R¹, R², R³ and R⁴ is selected from the group consisting of alkyl, substituted alkyl, halo, and alkoxy; and,
R⁷ is selected from the group consisting of phenyl, substituted phenyl, and anthracenyl; and
b) a metal precursor characterized by the general formula M(L)ₙ where M is a metal selected from groups 3-6 of the Periodic Table of Elements and Lanthanide elements of the Periodic Table of Elements, each L is a moiety that forms a covalent, dative or ionic bond with M; and n is 1, 2, 3, 4, 5, or 6.

10. The composition of claim 9, wherein the compound in part a) is characterized by a formula selected from the group consisting of 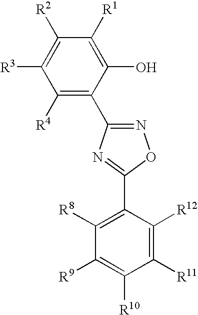
wherein
R¹, R², R³, and R⁴ are as defined in claim 9; and,
R⁸, R⁹, R¹⁰, R¹¹, R¹² are the same or different and are independently selected from the group consisting of hydrogen, alkyl, substituted alkyl, cycloalkyl, substituted cycloalkyl, heteroalkyl, substituted heteroalkyl, heterocycloalkyl, substituted heterocycloalkyl, aryl, substituted aryl, heteroaryl, substituted heteroaryl, alkoxy, aryloxy, halo, silyl, boryl, phosphino, amino, thioalkyl, thioaryl, nitro, and combinations thereof; and optionally two or more of R⁸, R⁹, R¹⁰, R¹¹, and R¹² may be joined to form a fused ring system having up to 50 atoms, not counting hydrogen atoms.

11. The composition of claim 9, wherein R¹ is selected from the group consisting of carbazolyl and substituted carbazolyl.

12. The composition of claim 11, wherein R⁷ is selected from the group consisting of substituted phenyl and anthracenyl.

13. The composition of claim 12, wherein R¹ is selected from the group consisting of carbazolyl and substituted carbazolyl; R³ is selected from the group consisting of halo and tBu; and R⁷ is selected from the group consisting of substituted phenyl and anthracenyl.

14. The composition of claim 9, wherein the ratio of ligand compound to the metal precursor is about two equivalents to one equivalent, and wherein the about two ligand equivalents are the same ligand.

15. A catalyst formed from the complex of claim 1 and an activator, combination of activators or an activating technique.

16. The catalyst of claim 15, wherein the catalyst is supported before or after activation.

17. A catalyst formed from the composition of claim 9 and an activator, combination of activators or an activating technique.

18. The catalyst of claim 18, wherein the catalyst is supported before or after activation.

19. A polymerization process comprising subjecting one or more monomers to polymerization conditions in the presence of a catalyst comprising the complex of claim 1 and an activator, combination of activators or an activating technique.

20. The process of claim 19, wherein the process is a copolymerization of ethylene and one or more α-olefins or cyclic olefin monomers.

21. The process of claim 20, wherein the one or more monomers is selected from the group consisting of ethylene, propylene, 1-butene, 1-hexene, 1-octene, 1-decene, styrene and combinations thereof.

22. The process of claim 19, wherein the process is a solution polymerization process conducted at a temperature greater than or equal to about 120° C.

23. A polymerization process comprising subjecting one or more monomers to polymerization conditions in the presence of a catalyst comprising the composition of claim 9 and an activator, combination of activators or an activating technique.

24. The process of claim 23, wherein the process is a copolymerization of ethylene and one or more α-olefins or cyclic olefin monomers.

25. The process of claim 24, wherein the one or more monomers is selected from the group consisting of ethylene, propylene, 1-butene, 1-hexene, 1-octene, 1-decene, styrene and combinations thereof.

26. The process of claim 23, wherein the process is a solution polymerization process conducted at a temperature greater than or equal to about 120° C.
